(19) United States
(12) Patent Application Publication
(10) Pub. No.: US 2007/0161552 A1
(43) Pub. Date: Jul. 12, 2007

(54) Use of low-dosage erythropoietin for stimulation of endothelial progenitor cells, for organ regeneration and for slowing the progression of end-organ damage

(76) Inventors: Ferdinand Hermann Bahlmann, Hannover (DE); Hermann Haller, Hannover (DE)

Correspondence Address: OSTROLENK FABER GERB & SOFFEN, 1180 AVENUE OF THE AMERICAS, NEW YORK, NY, 100368403, US

(21) Appl. No.: 10/586,896

(22) PCT Filed: Jan. 22, 2005
(86) PCT No.: PCT/EP05/00618

§ 371 Date: Oct. 16, 2006

(30) Foreign Application Priority Data
Jan. 23, 2004 (DE) 10 2004 004 509.7

(51) Int. Cl. A61K 38/18

(52) U.S. Cl. 514/12

(57) ABSTRACT

The present invention relates to the use of low-dosage erythropoietin for stimulation of physiological mobilization, proliferation and differentiation of endothelial progenitor cells, for stimulation of vasculogenesis, for therapy of diseases related to a dysfunction of endothelial progenitor cells, and for production of pharmaceutical compositions for treatment of such diseases as well as pharmaceutical compositions that include erythropoietin and other suitable active ingredients for stimulation of endothelial progenitor cells.

The present invention relates to the use of erythropoietin (EPO) especially in low doses, alone or in combination with other chemical, thermal, mechanical and biological agents, for stimulation of physiological mobilization, proliferation and differentiation of endothelial progenitor cells, for stimulation of vasculogenesis, for therapy of diseases related to a dysfunction of endothelial progenitor cells, and for production of pharmaceutical compositions for treatment of such diseases as well as pharmaceutical compositions that include erythropoietin and other suitable active ingredients for stimulation of endothelial progenitor cells as well as for organ protection, for organ regeneration, especially formation of new vessels and tissues, and for slowing the progression of organ damage.

The present invention also relates to the use of erythropoietin, especially in the inventive low doses, and/or suitable active ingredients for application, preferably topical, in cosmetic treatment, and therefore in the sense of “beauty care” for the human or animal body, especially for prevention or reduction of creases and wrinkles, for strengthening of the connective tissue, for protection and tightening of the skin, especially facial skin, against harmful environmental factors, and as makeup foundation. Furthermore, the inventive topical use of erythropoietin counteracts the formation and further development of age spots, refines the skin texture, supports the skin rejuvenation process and accelerates hair growth.

The present invention also relates to the use of erythropoietin, preferably in low doses, or in other words EPO, preferably dosed as defined in the section entitled “Inventive dosing of EPO” hereinafter, for production of a pharmaceutical composition that is suitable and designed for application in a manner adapted to the natural circadian rhythm of EPO in the human or animal body. In humans, endogenous erythropoietin production has its acro phase (daily maximum) in the late afternoon, and so the inventive administration of the low-dosage erythropoietin as defined in the foregoing preferably takes place in the morning, especially in the period from 6:00 to 10:00 a.m., in order in this way to achieve a maximum biological and therefore also therapeutic effect. Within this time period, the EPO can be administered as a single dose or as multiple doses. According to the invention, this use as a single dose or as multiple doses is proposed particularly preferably for all uses cited according to the present teaching, especially for cosmetic and therapeutic treatment of the human and animal body or cells.

According to the invention, it is provided in a further embodiment of the use of erythropoietin that endothelial progenitor cells will be applied simultaneously with other cell populations usable for cell therapy, after prior incubation with low-dosage erythropoietin in vitro, preferably in low doses, or local as well as systemic application of erythropoietin in vivo, preferably in low doses according to the invention, in order in this way to ensure that the tissue cells useable for cell therapy settle with sufficient binding to the vascular system.

The invention therefore also relates to the use of erythropoietin in vivo, preferably in low doses, preferably for morning application in a period from 06:00 to 10:00 a.m., during application of endothelial progenitor cells having at least one cell population usable for cell therapy, in order to improve the settling, with sufficient binding to the vascular system, of the cell population usable for cell therapy. The invention also relates to the use of erythropoietin in vitro, preferably in low doses, for incubation with endothelial progenitor cells and at least one cell population usable for cell therapy, in order to improve the settling, with sufficient binding to the vascular system, of the cell population usable for cell therapy.

The invention also relates to the use of erythropoietin especially in low doses, especially for production of a pharmaceutical composition or of a kit for prevention or treatment of diseases or for use during transplantation or implantation, in sequential, timed successive administration with at least one other chemical, thermal, mechanical or biological agent, especially a pharmacological active ingredient, for increasing the number and function of endothelial progenitor cells and/or for regeneration or slowing the progression of tissue damage.

The invention also relates to the use of use of erythropoietin, especially for production of a pharmaceutical composition or of a kit, for prevention or treatment of diseases or for use during transplantation or implantation, especially in low doses, for simultaneous administration of erythropoietin and at least one other chemical, thermal, mechanical or biological agent, for increasing the number and function of endothelial progenitor cells and/or for regeneration or slowing the progression of tissue damage.

The invention therefore relates to the preferably sequential, timed successive or simultaneous administration of low-dosage erythropoietin plus, in a preferred embodiment, one or more other pharmacological active ingredients, such as VEGF; GM-CSF, M-CSF, thrombopoietin, SDF-1, SCF, NGF, PIGF, an HMG coreductase inhibitor, an ACE inhibitor, an AT-1 inhibitor and an NO donor, in order in this way to increase the number and function of endothelial progenitor cells and/or to bring about regeneration or slowing of the progression of tissue damage. In this connection, the intention according to the invention is to influence the following sequence: A) quantitative and qualitative optimization of stem cells and/or endothelial progenitor cells in bone marrow or in specific tissue niches for stem cells; B) mobilization of stem cells and/or endothelial progenitor cells from bone marrow or other “stem cell” niches into peripheral blood; C) quantitative and qualitative optimization of stem cells and/or endothelial progenitor cells in peripheral blood and/or ex vivo under selective culture conditions, preferably cultures under hypoxic conditions with an oxygen concentration of 0.1% to 10%; D) homing of stem cells and/or endothelial progenitor cells to the damage site; E) adhesion and migration of stem cells and/or endothelial progenitor cells into the target tissue; F) neovascularization by endothelial progenitor cells.

The invention therefore also relates to the sequential, timed successive or simultaneous administration of erythropoietin, in low doses according to the invention, especially in vivo and in vitro, and if necessary also of one or more chemical, thermal, mechanical or biological agents, in order in this way to increase the number and function of endothelial progenitor cells and/or to bring about regeneration or slowing of the progression of tissue damage, an optional and preferable use being as described hereinabove, in a manner adapted to the natural circadian rhythm of endogenous EPO production, or in other words in an application form that is suitable and designed for administration in the period from 6:00 to 10:00 a.m.

The present invention also relates to the use of erythropoietin, in low doses according to the invention, for stimulation of physiological mobilization, proliferation and differentiation of endothelial progenitor cells, for stimulation of vasculogenesis, for therapy of diseases related to a dysfunction of endothelial progenitor cells, and for production of pharmaceutical compositions for treatment of such diseases as well as pharmaceutical compositions that include erythropoietin and other suitable active ingredients for stimulation of endothelial progenitor cells, of or for patients with a) a dysfunction of endothelial progenitor cells and b) with at least one cardiovascular risk factor such as hypertension, hypercholesterolemia, elevated asymmetric dimethylarginine (ADMA) levels, insulin resistance, hyperhomocysteinemia and c) at least one end-organ damage, such as left ventricular hypertrophy, microalbuminuria, cognitive dysfunction, increased thickness of the intima media in the carotid artery, proteinuria or a glomerular filtration rate (GFR) of <80 ml/min, especially 30, preferably 40 to 80 ml/min. Preferably the invention relates to the aforesaid use of low-dosage EPO in the aforesaid patient groups defined in a) to c) in an embodiment that is suitable and designed for undertaking application of EPO in a period from 6:00 to 10:00 a.m.

The vascular endothelium is a layer of cells that lines the blood vessels. The endothelium keeps the blood separate from other vascular layers. However, the endothelium is not merely a passive barrier, but it participates actively in regulation of vascular tone. This capability is also referred to as endothelium-dependent vasodilation. Because of its location, the endothelium is exposed at all times to hemodynamic stress and metabolic stress. In pathogenic conditions, such as high blood pressure, elevated LDL levels, elevated levels of asymmetric dimethylarginine, which is the endogenous inhibitor of NO synthetase, hypercholesterolemia, restricted renal function with a glomerular filtration rate of 30, preferably 40 to 80 ml/min, insulation resistance or high blood glucose, there frequently develop functional defects of the endothelium, potentially followed by morphologically detectable damage, such as formation of atherosclerotic plaques and/or further end-organ damage, such as left ventricular hypertrophy, microalbuminuria, proteinuria, neuropathies or microcirculation impairments. A very early sign of altered or reduced endothelial function or endothelial dysfunction is a decline of endothelium-dependent vasodilation.

In the case of coronary heart disease, or even when risk factors are present without coronary heart disease, especially hypertension, restricted renal function, hyperlipoproteinemia, hyperhomocysteinemia, insulin resistance or diabetes, endothelial function defects are manifested in decreased production of NO (=EDRF) and increased endothelin production. High plasma levels of endothelin lead to abnormal coalescence of cells, inflammation, vascular tumors and severe vascular stenoses. Endothelial function impairments are additionally characterized by increased production of adhesion molecules such as ICAM-1 and VCAM-1, whereby thrombocytes and monocytes adhere to an increased degree to the endothelium. This results in increased vasotonia. Thus a disequilibrium favoring vasoconstriction, adhesion, aggregation, coagulation, atherosclerosis and atherothrombosis develops in the most diverse systems. Even mental stress leads to measurable endothelial dysfunction, which can persist for as long as 4 hours.

Endothelial cells also participate in the formation of new blood vessels. Blood vessel formation is important in a large number of processes, such as embryogenesis, the female reproductive cycle, wound healing, tumor growth and neovascularization of ischemic regions. Originally, postnatal blood vessel formation, or in other words blood vessel formation after birth, was attributed mainly to angiogenic processes. By angiogenesis there will be understood the development of new blood vessels by sprouting of capillaries from a preexisting vascular system. During angiogenesis, the basement membrane surrounding the blood vessels is first destroyed by means of proteolytic enzymes, and the extracellular matrix in the perivascular space is fragmented. The angiogenic stimuli released thereby cause already existing differentiated endothelial cells to migrate toward the chemotactic stimulus, during which process they simultaneously proliferate and are transformed. New vascular loops with a capillary-type lumen are then formed by accretion of endothelial cells. Thereafter synthesis of a new basement membrane begins.

Recent investigations, however, show that the formation of new blood vessels in the adult organism depends not only on angiogenesis but also on vasculogenic mechanisms. By vasculogenesis there is understood formation of new vessels from endothelial progenitor cells undergoing differentiation in situ. The belief that vasculogenesis is confined to embryogenesis was refuted by the detection of endothelial progenitor cells (EPC) in peripheral blood of healthy humans and animals. By using animal models, it was proved that the endothelial progenitor cells derived from bone marrow participate actively in neovascularization. It was also shown that a specific CD34-positive subgroup of leukocytes expressing endothelium-specific antigens becomes established in ischemic regions. In addition, endothelial progenitor cells (EPC) that contribute significantly to the formation of blood vessels in the adult organism can be obtained in vitro from CD133+ and CD34+ cells (Asahara et al., Science, 275 (1997), 964-967; Crosby et al., Circ. Res., 87 (2000), 728-730; Gehling et al., Blood, 95 (2000), 3106-3112). It was also shown that injection of isolated CD34+ cells or cultivated endothelial progenitor cells accelerates restoration of blood flow in diabetic mice (Schatteman et al., J. Clin. Invest., 106 (2000), 571-578) and improves neovascularization in vivo (Asahara et al., Circ. Res., 85 (1999), 221-228; Crosby et al., Circ. Res., 87 (2000), 728-730; Murohara et al., J. Clin. Invest., 105 (2000), 1527-1536). Furthermore, it was shown that neovascularization induced by CD34+ cells improves cardiac function (Kocher et al., Nat. Med., 7 (2001), 430-436). Besides CD34+ cells, CD34-negative mononuclear blood cells can also be used as a source of endothelial progenitor cells by appropriate transdifferentiation.

However, the mechanisms underlying mobilization and differentiation of endothelial progenitor cells have not yet been fully explained. Molecular biological and cytobiological investigations indicate that various cytokines and angiogenic growth factors have stimulating effects on mobilization of endothelial progenitor cells in bone marrow. For example, it is known that proangiogenic factors such as VEGF and GM-CSF can increase the number of endothelial progenitor cells (Asahara et al., EMBO, J., 18 (1999), 3964-3972; Takahashi et al., Nat. Med., 5 (1999), 434-438). VEGF (vascular endothelial growth factor) is a protein that occurs in various isoforms and that binds to the two tyrosine kinase receptors VEGF-R1 (flt-1) and VEGF-R2 (flk-1), which occur, for example, on the surface of growing endothelial cells (Wemert et al., Angew. Chemie, 21 (1999), 3432-3435). Activation of VEGF receptors leads via the Ras-Raf-MAP kinase pathway to expression of proteinases and specific integrins on the surface of endothelial cells or endothelial progenitor cells, and finally to initiation of proliferation and migration of these cells toward the angiogenic stimulus. GM-CSF (granulocyte-macrophage colony-stimulating factor) is a cytokine that heretofore was known mainly for stimulation of white blood corpuscles, including neutrophils, macrophages and eosinophils. PIGF (placental growth factor) is known to stimulate the mobilization of endothelial progenitor cells but not proliferation thereof. From investigations by Llevadot et al. (J. Clin. Invest., 108 (2001), 399-405), it follows that HMG-CoA reductase inhibitors, especially statins, which are used as lipid-lowering medicaments and which reduce the morbidity and mortality of coronary disease, are able to mobilize endothelial progenitor cells. Dimmeler et al. (J. Clin. Invest., 108 (2001), 391-397) were able to show further that statins such as atorvastatin and simvastatin significantly improve the differentiation, in vitro and in vivo, of endothelial progenitor cells in mononuclear cells and CD34+ stem cells isolated from peripheral blood. For example, treatment of mice with statins led to an increased number of differentiated endothelial progenitor cells, and the statins exhibited just as strong an effect as that of VEGF.

The present invention is based on the technical problem of providing means and methods for improved stimulation of endothelial progenitor cells and for the therapy of disorders, which in particular are associated with a dysfunction of endothelial progenitor cells, and also of providing means and methods for protection and regeneration of different tissues.

The present invention solves this technical problem by teaching that erythropoietin and/or its derivatives, especially in low doses, can be used for stimulation of the physiological mobilization of endothelial progenitor cells, the proliferation of endothelial progenitor cells, the differentiation of endothelial progenitor cells to endothelial cells and/or the migration of endothelial progenitor cells toward an angiogenic or vasculogenic stimulus in a human or animal body. The inventive stimulation of the mobilization and/or differentiation of endothelial progenitor cells represents an important new therapeutic strategy for increasing postnatal neovascularization, especially vasculogenesis, and for treating diseases associated with a dysfunction of endothelial progenitor cells and/or endothelial cells, as well as for protection and regeneration of different tissues damaged by chemical, thermal, mechanical and biological agents.

The present invention also solves this technical problem by teaching the use of low-dosage erythropoietin and/or its derivatives for the therapy of diseases or pathological states associated with a dysfunction of endothelial progenitor cells and/or endothelial cells.

Furthermore, the present invention also solves this technical problem by teaching the use of low-dosage erythropoietin and/or its derivatives for protection and regeneration of different tissue types in diseased condition, or for pathological states associated with a dysfunction of the respective tissue function.

The underlying technical problem is also solved by the sequential, timed successive or simultaneous administration of low-dosage erythropoietin plus one or more other chemical, thermal, mechanical or biological agents.

The invention therefore relates in particular to the following embodiments A) to K), individually and/or in combination:

A) The use of erythropoietin, preferably for production of a pharmaceutical composition, for prevention or treatment of diseases, wherein the erythropoietin or/and the pharmaceutical composition is suitable and designed for morning application to a human or animal body in the period from 6:00 to 10:00 a.m.

B) The use of erythropoietin, preferably in combination with embodiment A), especially for production of a pharmaceutical composition, for prevention or treatment of diseases, wherein the pharmaceutical composition in its low doses is suitable and designed for prevention or treatment of a human or animal patient exhibiting a) at least one dysfunction of endothelial progenitor cells, b) at least one cardiovascular risk factor such as hypertension, hypercholesterolemia, insulin resistance, elevated ADMA levels or hyperhomocysteinemia and c) at least one end-organ damage such as left ventricular hypertrophy, microalbuminuria, cognitive dysfunction, increased thickness of the intima media in the carotid artery, proteinuria or a glomerular filtration rate of <80 ml/min, preferably 30 to 80 ml/min.

C) The use of erythropoietin, preferably in combination with embodiment A), B) or A) and B), for cosmetic treatment of the human or animal body, especially for treatment of wrinkles, for strengthening of the connective tissue, for protection and tightening of the skin, for protection against harmful environmental effects, for treatment of age spots, for acceleration of reepithelialization, for acceleration of hair growth and/or as makeup foundation.

D) The use of erythropoietin, preferably in combination with embodiment A), B) or A) and B), for production of a cosmetic preparation, especially for topical application, for cosmetic treatment of the human or animal body, especially for treatment of wrinkles, for strengthening of the connective tissue, for protection and tightening of the skin, for protection against harmful environmental effects, for treatment of age spots, for acceleration of reepithelialization, for acceleration of hair growth and/or as makeup foundation.

E) The use of erythropoietin, preferably in combination with one or more of embodiments A), B), C) or D), and/or a mixture of endothelial progenitor cells with at least one cell population usable for cell therapy, for production of a pharmaceutical composition containing erythropoietin and a mixture of endothelial progenitor cells with at least one cell population usable for cell therapy, for regeneration of tissues or vessels in a human or animal body, wherein the mixture has been brought into contact with erythropoietin in vitro prior to application.

F) The use of erythropoietin, preferably in combination with one or more of embodiments A), B), C), D) or E), and/or a mixture of endothelial progenitor cells with at least one cell population usable for cell therapy, for production of a pharmaceutical composition containing erythropoietin and/or a mixture of endothelial progenitor cells with at least one cell population usable for cell therapy, for regeneration of tissues or vessels in a human or animal body, wherein erythropoietin is administered to the animal or human body before, after or simultaneously with application of the mixture.

G) The use of erythropoietin, preferably in combination with one or more of the embodiments according to A) to F), and/or at least one chemical, thermal, mechanical or biological agent, especially a pharmacological active ingredient, for production of a pharmaceutical composition or of a kit containing erythropoietin and the at least one chemical, thermal, mechanical or biological agent, for prevention or treatment of diseases, wherein the pharmaceutical composition or the kit is suitable and designed for sequential, timed successive or simultaneous application of the erythropoietin with the at least one chemical, thermal, mechanical or biological agent. The invention therefore also relates to the use of erythropoietin in the manner indicated hereinabove under G, wherein the mechanical agents are endoprostheses, preferably implantation supports for teeth, bones or ligament/tendon replacements. The invention also relates to the use of erythropoietin in the manner indicated hereinabove under G), wherein the biological agents are solid organs such as liver, kidneys, heart, pancreas or skin. In this connection, hair implants will also be understood as biological agents. In a particularly preferred embodiment, the present invention therefore relates to the use of erythropoietin for production of a pharmaceutical composition or of a kit for systemic or local application at the implantation site of a biological agent of the foregoing type or of an endoprosthesis, especially an implantation support for a tooth, tooth replacement, tooth implant, bone replacement, bone implant, for example hip joint prosthesis, ligament/tendon replacements, for example cruciate ligament, wherein the erythropoietin is applied systemically or locally prior to implantation of the said biological or mechanical agent, or in other words, for example, the endoprosthesis, for example some weeks prior to implantation, after which implantation is performed. In a further embodiment, it is also provided that implantation of the said biological or mechanical agent, such as the endoprosthesis, be undertaken simultaneously with the use of erythropoietin. In a further embodiment, it is provided that the erythropoietin be administered after implantation of the said endoprosthesis or of the mechanical or biological agent. According to these embodiments, the tissue or the body structure in which the implant such as a tooth or bone prosthesis will be implanted is mobilized or conditioned, thus enabling considerably better and thus faster integration of the biological or mechanical agent, such as an implant, for example by growth onto or into the body structure.

H) The use of erythropoietin according to one or more of the embodiments according to A) to G), wherein the pharmaceutical composition does not lead to any increase of the hematocrit during application in the human or animal body, especially not more than 10% of the value of the hematocrit prior to application of the erythropoietin.

I) The use of erythropoietin according to one or more of embodiments according to A) to H) in a pharmaceutical composition, wherein the erythropoietin is suitable and designed for the said prevention, treatment or therapy, in a low dose that cannot activate erythropoiesis, especially in a dose of 0.001 IU/kg of body weight per week, up to 90, especially 50 IU/kg of body weight per week.

K) The use of erythropoietin according to one or more of the embodiments according to A) to I), wherein the disease can be hypercholesterolemia, diabetes mellitus, insulin resistance, endothelium-mediated chronic inflammatory disorders, endotheliosis including reticuloendotheliosis, atherosclerosis, age-related cardiovascular disease, ischemic disorders of the extremities, preeclampsia, Raynaud's disease, hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, pregnancy-induced hypertension, chronic or acute renal failure, especially terminal renal failure, renal function restrictions with glomerular filtration rates of 30 to 80 ml/min, microalbuminuria, proteinuria, conditions with elevated ADMA levels or wounds and sequelae thereof.

The invention also relates to the production of a kit containing erythropoietin, endothelial progenitor cells and at least one cell population usable for cell therapy, wherein the erythropoietin is preferably present in low dose.

According to the invention, it has surprisingly been found that treatment with low-dosage erythropoietin leads to physiological mobilization of endothelial progenitor cells, wherein the number of circulating endothelial progenitor cells is increased and differentiation thereof is induced. In addition, functional deficits of the endothelial progenitor cells that occur under certain pathological conditions are compensated. According to the invention, it has been shown that the number of circulating stem cells in patients with chronic renal disorder in the terminal stage is just as high as in healthy subjects, but in these patients they have lost the ability to differentiate to endothelial cells via endothelial progenitor cells. Thus the number of cells capable of adhesion and exhibiting an endothelial cell phenotype is distinctly reduced in patients with chronic renal disorder compared with healthy subjects (de Groot et al., Kidney Int. 2004;66:641-6). This functional decline of endothelial progenitor cells can already be seen in moderate restriction of the renal function with a glomerular filtration rate of 30, preferably 40 to 80 ml/min. According to the invention, it has now been found that the number of circulating stem cells increases significantly by more than 50% after treatment with low-dosage erythropoietin according to the invention, not only in these patients but also in patients and/or subjects with healthy kidneys. In particular, the number of cells that develop an endothelial phenotype increases dramatically. As was demonstrated by means of a functional cell culture test, the impaired adhesion ability of the endothelial progenitor cells in patients with chronic renal disorder, characterized by a glomerular filtration rate of 30, preferably 40 to 80 ml/min, is increased by a factor of three by the low-dosage erythropoietin treatment. In subjects and/or patients with healthy kidneys, it is increased by a factor of two to three. The adhesion ability of endothelial progenitor cells undergoing differentiation and of endothelial cells is one of the basic prerequisites for the formation of new tissues and/or vessels. In this way erythropoietin is able to induce neovascularization, especially vasculogenesis, in tissues or organs, of which kidneys are a particular example, in which corresponding vasculogenic or angiogenic stimuli are released.

According to the invention, low-dosage erythropoietin can be used to stimulate physiological mobilization of endothelial progenitor cells, proliferation of endothelial progenitor cells, differentiation of endothelial progenitor cells to endothelial cells and/or migration of endothelial progenitor cells toward a vasculogenic or angiogenic stimulus in a human or animal body, especially an adult organism. According to the invention, low-dosage erythropoietin can therefore be employed advantageously to stimulate the formation of new vessels by vasculogenesis in tissues or organs in which pathological vascular changes are present. In addition, the formation of endothelial tissue can also be induced by virtue of the stimulation of endothelial progenitor cells by low-dosage erythropoietin. According to the invention, low-dosage erythropoietin can therefore also be employed to treat diseases of the human or animal body that are associated with a dysfunction of endothelial progenitor cells and/or endothelial cells. Patient populations exhibiting such a dysfunction usually have cardiovascular risk factors such as hypertension, hypercholesterolemia, insulin resistance, hyperhomocysteinemia, elevated ADMA levels, and end-organ damage such as left ventricular hypertrophy, microalbuminuria, proteinuria or a glomerular filtration rate (GFR) of 30, preferably 40 to 80 ml/min.

The invention also relates to the use of low-dosage erythropoietin for protection and regeneration of tissue whose function has been jeopardized by the action of chemical, thermal, mechanical or biological agents. According to the invention, topical application of low-dosage erythropoietin also relates to prevention and reduction of already existing wrinkles of the skin, especially of the facial skin, to protection of the skin and to reduction of age spots. According to the invention, such use of low-dosage erythropoietin or of a derivative can take place sequentially, in timed succession or simultaneously with one or more other chemical, thermal, mechanical or biological agents. According to the invention, low-dosage erythropoietin can be therapeutically used in a manner adapted to its circadian rhythm, in order in this way to achieve a maximum biological effect. In a preferred embodiment according to the invention, endothelial progenitor cells are applied simultaneously with other cell populations usable for cell therapy, after prior incubation with low-dosage erythropoietin in vitro and/or local as well as systemic application of low-dosage erythropoietin in vivo, in order in this way to ensure that the tissue cells usable for cell therapy settle with sufficient binding to the vascular system.

In connection with the present invention, there will be understood by “erythropoietin” or “EPO” a substance that, in appropriately high dosage, controls the growth, differentiation and maturation of stem cells via erythroblasts to erythrocytes.

Erythropoietin is a glycoprotein having 166 amino acids, three glycosylation sites and a molecular weight of about 34,000 Da. During EPO-induced differentiation of erythrocyte progenitor cells, globin synthesis is induced, synthesis of the heme complex is augmented and the number of ferritin receptors is increased. Thereby the cell can take up more iron and synthesize functional hemoglobin. In mature erythrocytes, hemoglobin binds oxygen. Thus the erythrocytes and the hemoglobin contained therein play a key role in supplying oxygen to the organism. These processes are initiated through the interaction of EPO with an appropriate receptor on the cell surface of the erythrocyte progenitor cells (Graber and Krantz, Ann. Rev. Med. 29 (1978), 51-56).

The term “erythropoietin” used here includes EPO of every origin, especially human or animal EPO. The term used here encompasses not only the naturally occurring, or in other words wild-type forms of EPO, but also its derivatives, analogs, modifications, muteins, mutants or others, as long as they exhibit the biological effects of wild-type erythropoietin.

In connection with the present invention, there will be understood by “derivatives” functional equivalents or derivatives of erythropoietin that, while retaining the basic erythropoietin structure, are obtained by substitution of one or more atoms or molecular groups or residues, especially by substitution of sugar chains such as ethylene glycol, and/or whose amino acid sequences differ from that of the naturally occurring human or animal erythropoietin protein in at least one position but essentially have a high degree of homology at the amino acid level and comparable biological activity. Erythropoietin derivatives such as can be employed, for example, in the present invention are known from WO 94/25055, EP 0148605 B1 or WO 95/05465, among other sources.

“Homology” means especially a sequence identity of at least 80%, preferably at least 85% and particularly preferably at least more than 90%, 95%, 97% and 99%. The term “homology” known by the person skilled in the art thus refers to the degree of relationship between two or more polypeptide molecules. This is determined by the agreement between the sequences. Such agreement can mean either identical agreement or else a conservative exchange of amino acids.

According to the invention, the term “derivative” also includes fusion proteins, in which functional domains of another protein are present on the N-terminal part or on the C-terminal part. In one embodiment of the invention, this other protein may be, for example, GM-CSF, VEGF, PIGF, a statin or another factor that has a stimulating effect on endothelial progenitor cells. In a further embodiment of the invention, the other protein may also be a factor that has a stimulating effect on differentiated endothelial cells, for example angiogenin, VEGF (vascular endothelial growth factor) or bFGF (basic fibroblast growth factor). Regarding bFGF and VEGF, it is known that these growth factors exert a strong mitogenic and chemotactic activity on endothelial cells.

The differences between an erythropoietin derivative and native erythropoietin may arise, for example, through mutations such as deletions, substitutions, insertions, additions, base exchanges and/or recombinations of the nucleotide sequences coding for the erythropoietin amino acid sequences. According to the invention, (EPO-) alpha, (EPO-) beta, Aranesp (darbepoetin alfa) or CERA (continuous erythropoietin receptor antagonist) are preferably used as erythropoietin. Obviously such differences can also be naturally occurring sequence variations, such as sequences from another organism or sequences that have mutated naturally, or mutations introduced selectively into the nucleic acid sequences coding for erythropoietin, using common means known in the art, such as chemical agents and/or physical agents. In connection with the invention, therefore, the term “derivative” also includes mutated erythropoietin molecules, or in other words erythropoietin muteins.

According to the invention, peptide or protein analogs of erythropoietin may also be used. In connection with the present invention, the term “analogs” includes compounds that do not have any amino acid sequence identical to the erythropoietin amino acid sequence but have a three-dimensional structure greatly resembling that of erythropoietin, so that they have comparable biological activity. Erythropoietin analogs may be, for example, compounds that contain, in a suitable conformation, the amino acid residues responsible for binding of erythropoietin to its receptors, and that are therefore able to simulate the essential surface properties of the erythropoietin binding region. Compounds of this type are described, for example, in Wrighton et al., Science, 273 (1996), 458. The EPO used according to the invention can be produced in various ways, for example by isolation from human urine or from the urine or plasma (including serum) of patients suffering from aplastic anemia (Miyake et al., J.B.C. 252 (1977), 5558). As an example, human EPO can also be obtained from tissue cultures of human renal cancer cells (JA Unexamined Application 55790/1979), from human lymphoblast cells, which have the ability to produce human EPO (JA Unexamined Application 40411/1982), and from a hybridoma culture obtained by cell fusion of a human cell line. EPO can also be produced by methods of gene technology, using suitable DNA or RNA coding for the appropriate amino acid sequence of EPO to produce the desired protein by genetic engineering, for example in a bacterium, in a yeast, or in a plant, animal or human cell line. Such methods are described, for example, in EP 0148605 B2 or EP 0205564 B2 and EP 0411678 B1.

The present invention relates in particular to the use of low-dosage erythropoietin and/or derivatives thereof for stimulation of physiological mobilization of endothelial progenitor cells, proliferation of endothelial progenitor cells, differentiation of endothelial progenitor cells to endothelial cells and/or migration of endothelial progenitor cells toward a vasculogenic or angiogenic stimulus in a human or animal body, especially an adult organism.

The invention also relates to sequential use of low-dosage erythropoietin and at least one further suitable chemical, thermal, mechanical or biological agent or active ingredient, especially a pharmacological active ingredient, that increases the function and number of endothelial progenitor cells and also potentiates the effect of low-dosage erythropoietin as regards organ protection and regeneration.

Furthermore, the invention therefore relates preferably to sequential, timed successive or simultaneous administration of low-dosage erythropoietin plus one or more other pharmacological active ingredients, such as VEGF; GM-CSF, M-CSF, thrombopoietin, SCF, SDF-1, NGF, PIGF, an HMG coreductase inhibitor, an ACE inhibitor, an AT-1 inhibitor and an NO donor, in order in this way to increase the number and function of endothelial progenitor cells and/or to bring about regeneration or slowing of the progression of tissue damage. In this connection, the intention according to the invention is to influence the following sequence: A) quantitative and qualitative optimization of stem cells and/or endothelial progenitor cells in bone marrow or in specific tissue niches for stem cells; B) mobilization of stem cells and/or endothelial progenitor cells from bone marrow or other “stem cell” niches into peripheral blood; C) quantitative and qualitative optimization of stem cells and/or endothelial progenitor cells in peripheral blood and/or ex vivo under selective culture conditions, preferably cultures under hypoxic conditions with an oxygen concentration of 0.1% to 10%; D) homing of stem cells and/or endothelial progenitor cells to the damage site; E) adhesion and migration of stem cells and/or endothelial progenitor cells into the target tissue; F) neovascularization by endothelial progenitor cells.

The present invention therefore relates to application, simultaneously or at different times, of endothelial progenitor cells and one or more cell populations usable for cell therapy, especially hepatocytes, myocytes, cardiomyocytes or island transplants, after prior incubation with low-dosage erythropoietin in vitro and/or local as well as systemic application of low-dosage erythropoietin in vivo, thus improving and accelerating the function, settling, vascularization and connection to the blood circulation of the recipient of these cell populations used for cell therapy.

The present invention relates to the use of erythropoietin, especially in low doses, or suitable active ingredients for topical application in the sense of “beauty care”, especially for prevention or timely reduction of creases and wrinkles, strengthening of the connective tissue, protection and tightening of the skin, especially facial skin, against harmful environmental factors, and as makeup foundation. Furthermore, the topical use of erythropoietin counteracts the formation and further development of age spots, refines the skin texture and supports the skin rejuvenation process, especially reepithelialization. In addition, erythropoietin accelerates hair growth.

The present invention also relates to the use of low-dosage erythropoietin for production of a pharmaceutical composition that is suitable and designed for application in a manner adapted to the circadian endogenous rhythm of erythropoietin. Endogenous erythropoietin production has its acro phase (daily maximum) in the late afternoon, and so the administration of the low-dosage erythropoietin preferably takes place in the morning, especially between 6:00 and 10:00 a.m., in order in this way to achieve a maximum biological, therapeutic or cosmetic effect.

The present invention relates to the use of low-dosage erythropoietin for stimulation of physiological mobilization, and/or for proliferation and differentiation of endothelial progenitor cells, and/or for stimulation of vasculogenesis, and/or for therapy of diseases related to a dysfunction of endothelial progenitor cells, and/or for production of pharmaceutical compositions for treating such diseases and of pharmaceutical compositions that include erythropoietin and other suitable active ingredients for stimulation of endothelial progenitor cells, in patients with a) a dysfunction of endothelial progenitor cells, and b) at least one cardiovascular risk factor such as hypertension, hypercholesterolemia, insulin resistance, hyperhomocysteinemia, elevated ADMA levels and c) at least one end-organ damage such as left ventricular hypertrophy, microalbuminuria, cognitive dysfunction, increased thickness of the intima media in the carotid artery, proteinuria or a glomerular filtration rate (GFR) of less than 80 ml/min, especially 30, preferably 40 to 80 ml/min.

In a preferred embodiment, the invention also relates to sequential, timed successive or simultaneous administration of low-dosage erythropoietin as well as one or more other chemical, thermal, mechanical and biological agents, in order in this way to increase the number and function of endothelial progenitor cells and/or to bring about regeneration or slowing of the progression of tissue damage. Such mechanical agents can be endoprostheses, preferably implantation supports for teeth, bones or ligament/tendon replacements. Furthermore, the biological agents can be solid organs such as liver, kidneys, heart, pancreas or skin, or even hair implants. The invention therefore provides that EPO, especially in low doses, will be used so that mechanical agents such as endoprostheses or biological agents implanted simultaneously, subsequently or beforehand can grow or be integrated better, faster and more efficiently into the surrounding body structure. The invention therefore also relates to the use of erythropoietin for production of a pharmaceutical composition or of a kit for improving, especially for promoting or accelerating, integration of biological agents or endoprostheses into surrounding body structures, especially of teeth, tooth replacements, tooth implants or other endoprostheses, such as bone replacements, bone implants, especially hip joint prostheses or ligament/tendon replacements, such as cruciate ligaments. In this connection, it can be provided if necessary that the erythropoietin will be used together with cell populations suitable for cell therapy and/or endothelial progenitor cells. In the aforesaid use of erythropoietin for production of a pharmaceutical composition or of a kit for improving, especially for promoting or accelerating, integration of biological or mechanical agents into target structures, especially target tissue, target bones or target cartilage of a patient, it can be provided in a further preferred embodiment that the mechanical agents to be used will be made, for example, of steel, ceramic, plastic or another matrix material. In addition, it can be provided that osteoblasts, cells having osteogenic potential, thrombocytes, blood cells or similar agents can be used as cell populations suitable for cell therapy in the present application. In a further preferred embodiment, it can be provided that the mechanical agent in particular to be used will be contained in the pharmaceutical composition or in the pharmaceutical kit together with organic adhesive, such as a fibrin glue.

In connection with the present invention, there will be understood as “endothelial progenitor cells” (EPC) cells that circulate in the bloodstream and have the ability to differentiate to endothelial cells. The endothelial progenitor cells occurring during embryonic development are angioblasts. The endothelial progenitor cells occurring in the adult organism are angioblast-like cells, which can be obtained from mononuclear cells, especially CD34+ to CD14+ monocytes, and/or CD34+ stem cells that have been isolated from peripheral blood.

In connection with the present invention, there will be understood by “mobilization” or “physiological mobilization” the process of activating stem cells and/or progenitor cells from the bone marrow or from alternative “stem cell” niches by growth factors, wherein the stem cells or progenitor cells enter the bloodstream, especially the peripheral blood.

In connection with the present invention, there will be understood by “proliferation” the ability of cells to become larger and subsequently divide into two or more daughter cells. The EPO-mediated stimulation of endothelial progenitor cells thus relates in particular to the number and thus the dividing behavior of endothelial progenitor cells.

In connection with the present invention, there will be understood by “differentiation” of endothelial progenitor cells the development of mononuclear cells originating from the bone marrow or specific tissue niches via endothelial progenitor cells into endothelial cells. By “endothelial cells” there will be understood the cells that form the endothelium, or in other words the monolayer cellular lining of vessels and serous cavities. Endothelial cells are characterized in that they release vasoactive substances, for example vasodilating substances such as EDRF (endothelial derived relaxing factor) or constricting substances such as endothelin, factors for inhibition or activation of blood clotting and factors for regulation of vascular permeability. Endothelial cells also synthesize components of the subendothelial connective tissue, especially type IV and V collagens, cell adhesion proteins such as laminin, fibronectin and thrombospondin, growth factors, for example for smooth muscle cells, and factors for the formation of new vessels.

In connection with the present invention, there will be understood by “migration” of endothelial progenitor cells the fact that the endothelial progenitor cells present in the bloodstream migrate toward a vasculogenic or angiogenic stimulus and become concentrated in the region of the vasculogenic or angiogenic stimulus. By “vasculogenic stimulus” there will be understood a chemical stimulus in a tissue or blood vessel of a human or animal body that acts specifically on endothelial progenitor cells and brings about migration thereof to that site in the body from which the chemical stimulus originates. In this way, the vasculogenesis process is induced by the vasculogenic stimulus. By “angiogenic stimulus” there will be understood a chemical stimulus in a tissue or blood vessel of a human or animal body that acts specifically on differentiated endothelial cells and brings about migration thereof to that site in the body from which the chemical stimulus originates. In this way, induction of angiogenesis is induced by the angiogenic stimulus.

In a further embodiment of the invention, there is provided the use of low-dosage erythropoietin and/or derivatives thereof for increasing the adhesion ability of endothelial progenitor cells undergoing differentiation. According to the invention erythropoietin is used in particular for improving the adhesion ability or in other words the cell-to-cell adhesion of endothelial progenitor cells. The adhesion of endothelial progenitor cells undergoing differentiation or differentiated endothelial cells is one of the basic prerequisites for the formation of new vessels or of new endothelial tissue. Cell adhesion is mediated by protein molecules.

The present invention also relates to the use of low-dosage erythropoietin for stimulation of the formation of new vessels, especially stimulation of vasculogenesis. In connection with the present invention, there will be understood by “vasculogenesis” the formation of new vessels from endothelial progenitor cells undergoing differentiation in situ. According to the invention, therefore, it is ensured by the use of low-dosage erythropoietin that endothelial progenitor cells can participate to an increased degree in formation of new vessels or in local formation of new vessels to restore damaged vascular regions. According to the invention, therefore, it is provided that the use of low-dosage erythropoietin and/or its derivatives will promote formation of new blood vessels and/or replacement of damaged vascular regions through local formation of new blood vessels.

In a further embodiment of the invention, there is provided the use of low-dosage erythropoietin and/or derivatives thereof for stimulation of endothelial progenitor cells for formation of endothelial tissue.

In a particularly preferred embodiment of the invention, there is provided the use of low-dosage erythropoietin and/or derivatives thereof for the therapy of pathological states or diseases of the human or animal body associated with a dysfunction of endothelial progenitor cells, or of sequelae thereof.

In connection with the present invention, there will be understood by “diseases”, “pathological states” or “disorders” impairments of vital processes in organs or in the entire organism, resulting in subjectively experienced or objectively detectable physical, emotional or mental changes. According to the invention, these diseases are associated in particular with a dysfunction of endothelial progenitor cells, or in other words diseases that either are the result of such a dysfunction of these cells or are mediated by these cells. Also according to the present invention, there will be understood by “diseases”, “pathological states” or “disorders” impairments of vital processes in organs or in the entire organism that can be arrested or in particular slowed in their progression by administration of low-dosage erythropoietin or suitable active ingredients. By “sequelae” there will be understood secondary diseases, or in other words a second disorder occurring in addition to a primary clinical condition.

In connection with the present invention, there will be understood by “dysfunction” of endothelial progenitor cells an impairment of essential cell functions such as metabolic activities, response to stimuli, motility, dividing behavior or differentiation behavior of these cells. A dysfunction of endothelial progenitor cells may mean, for example, that these cells proliferate not at all or only inadequately. Since the proliferation of endothelial progenitor cells is stimulated by the use of erythropoietin, the deficient dividing behavior both of endothelial progenitor cells and of already differentiated endothelial cells can thereby be compensated and the number of endothelial progenitor cells or endothelial cells increased. Dysfunction of endothelial progenitor cells may consist, for example, of impaired ability of these cells to differentiate to endothelial cells. A dysfunction of endothelial progenitor cells may also be caused by their impaired adhesion ability and/or their impaired ability to migrate toward an angiogenic or vasculogenic stimulus. Such dysfunctions of endothelial progenitor cells may lead, for example, to impairment or prevention of the formation of new endothelial tissue and/or of vasculogenesis. A dysfunction of endothelial progenitor cells may also have a pathogenic cause, for example due to hypertension, hyperlipoproteinemia, elevated ADMA blood levels, uremia or diabetes. The dysfunction of endothelial progenitor cells may be manifested, for example, by reduced production of NO (=EDRF) by NO synthases (NOS) from L-arginine, increased endothelin production and/or increased production of adhesion molecules such as ICAM-1 and VCAM-1.

According to the invention, the diseases associated with a dysfunction of endothelial progenitor cells are in particular hypercholesterolemia, diabetes mellitus, insulin resistance, endothelium-mediated chronic inflammatory disorders such as vascular inflammations, endotheliosis including reticuloendotheliosis, atherosclerosis, age-related cardiovascular disease, ischemic disorders of the extremities, Raynaud's disease, preeclampsia, pregnancy-induced hypertension, chronic or acute renal failure, especially terminal renal failure, renal function restrictions with glomerular filtration rates of 30 to 80 ml/min, preferably 40 to 80 ml/min, microalbuminuria, proteinuria, elevated ADMA levels, wound healing and sequelae thereof.

“Hypercholesterolemia” is characterized by elevated concentrations of cholesterol in the blood. By far the most frequent form of primary hypercholesterolemia is polygenic hypercholesterolemia. Secondary hypercholesterolemia frequently occurs in diabetes mellitus, nephrotic syndrome, hypothyroidism and hepatic disorders.

“Diabetes mellitus” encompasses various forms of glucose metabolism impairments having different etiologies and symptoms. In particular, the AGE-RAGE system is responsible for the development of diabetic complications related to vascular systems. AGEs (advanced glycation end products) are formed by a series of complex reactions following prolonged exposure of proteins or lipids to reducing sugars, for example glucose. The formation of AGEs takes place during the normal aging process and to an increased extent in diabetes mellitus and Alzheimer's disease. Binding of AGEs leads to oxidative stress, activation of the NF-κB transcription factor and thus an impairment of endothelial homeostasis.

By “insulin resistance” there will be understood impaired signal transmission in various body cells, which ignore the physiological signal cascade of insulation. Affected patients therefore lack normal glucose metabolism.

“Endothelium-mediated chronic inflammatory disorders” are disorders or conditions of a human or animal body that are caused by a defense response of the organism and its tissues to harmful stimuli, wherein certain signal molecules alter the properties of endothelial cells, with the result that, in interaction with the activation of other cell types, leukocytes remain adhering to endothelial cells, finally penetrating into the tissue and causing inflammation therein. One example of endothelium-mediated inflammation is leukocytic vasculitis. A central role in activation of an endothelium-mediated inflammatory event is played by the NF-κB transcription factor. Another system leading to the development of endothelial cell-mediated chronic inflammations is the AGE-RAGE system.

By “endotheliosis” there will be understood degenerative and proliferative endothelial changes during non-thrombopenic purpura. By “reticuloendotheliosis” there will be understood diseases of the reticulohistiocytic system, such as reticulum, reticulosis, reticulohistiocytosis and Hand-Schüller-Christian disease.

By “Raynaud's disease” there will be understood episodically occurring ischemic states caused by vasoconstriction, or in other words vascular spasms, usually in the arteries of the fingers. Primary Raynaud's disease is a purely functional impairment of the small vessels supplying the distal parts of the extremities, whereas secondary Raynaud's disease accompanies another disease such as vascular inflammation.

“Preeclampsia” is an endothelial and vascular disease of the maternal organism, apparently caused by endotheliotropic substances from the placenta. Preeclampsia is a multisystem disorder that may lead to functional impairments of numerous organs and be manifested by diverse symptoms. The circulatory impairments typical of the disorder result from increased vascular resistance, which can vary locally in severity. For preeclampsia it has been confirmed that an endothelial dysfunction is the central component of the pathogenesis.

In connection with the present invention, by “renal failure” there will be understood the restricted ability of the kidneys to excrete substances normally contained in the urine. In advanced stages, the ability to regulate the electrolyte, water and acid-base balance is also lost. Terminal renal failure is characterized by collapse of the excretory and endocrine function of the kidneys.

According to the invention, renal failure may be acute renal failure, which is also referred to as acute renal insufficiency, shock kidney or shock aneuria. Acute renal failure is characterized by sudden partial or total loss of the excretory function of the kidneys as a result of kidney damage that is usually reversible. The causes may be hypoperfusion due to hypovolemia, hypotension and dehydration resulting from blood losses (polytrauma, gastrointestinal or postpartum bleeding, major surgical procedures on the heart, vessels, abdomen or prostate), shock (myocardial infarction, embolism), serious infections (sepsis, peritonitis, cholecystitis), hemolysis (hemolytic-uremic syndrome, paroxysmal hemoglobinuria, transfusion reaction), myolysis (crush syndrome, rhabdomyolysis, myositis, burns), water and electrolyte losses (massive vomiting, diarrhea, excessive sweating, ileus, acute pancreatitis). Further causes may be nephrotoxins such as exogenous toxins, for example aniline, glycol compounds, methanol and the like, or endogenous toxins, for example myoglobin and oxalates. Further causes of acute renal failure are renal disorders, for example inflammatory nephropathies or rejection reactions following kidney transplantation. Acute renal failure may also be caused by urinary retention following obstruction of the urine flow. The inventive treatment of acute renal failure with erythropoietin, preferably in low doses, leads according to the invention to prevention or at least diminution of the progression of acute renal failure.

According to the invention, renal failure may also be chronic renal failure. Causes of chronic renal failure are vascular, glomerular and tubulointerstitial kidney disorders, infections and congenital or acquired structural defects. Causes of chronic renal failure include chronic glomerulopathy, chronic pyelonephritis, analgesic nephropathy, obstructive uropathy, arteriosclerosis and arteriolosclerosis. The terminal stage of chronic renal failure is uremia. The inventive treatment of chronic renal failure with low-dosage erythropoietin leads according to the invention to diminution of the progression of chronic renal failure.

In particular, the invention therefore relates to the use of EPO, preferably in low doses, for production of a drug for prevention, diminution or slowing of the damage to kidney tissue and/or for regeneration of damaged kidney tissue in cases of acute or chronic renal failure.

According to the invention, there will be understood by renal function restriction conditions in which the glomerular filtration rate has already slowed to less than 80 ml/min. Renal function restriction therefore relates to the early phase of glomerular, tubulointerstitial and vascular kidney disorders. The inventive treatment of renal function restrictions with low-dosage erythropoietin leads according to the invention to diminution of the progression or to regeneration of the beginning kidney tissue and/or function damage.

In connection with the present invention, there will be understood by “microalbuminuria” a clinical picture in which affected patients exhibit unphysiological excretion of albumin in the urine in excess of 30 mg per 24 hours. This increased albumin excretion is an early sign of the beginning of renal function deterioration, and is a consequence of the first pathological transformation processes in the kidneys, accompanied by structural alterations of the kidney architecture.

In connection with the present invention, there will be understood by “proteinuria” a clinical picture in which affected patients exhibit unphysiological excretion of proteins in the urine in excess of 150 mg per 24 hours. This increased protein excretion via the urine (>150 mg per 24 hours) is considered to be pathological, requiring further medical investigation and therapy.

In connection with the present invention, there will be understood by “high ADMA levels” a clinical picture in which affected patients exhibit an unphysiologically high ADMA blood concentration in excess of 1.3 μmol/l. This elevated ADMA concentration is associated with an endothelial dysfunction and is a consequence of metabolic dysfunctions in the processes of degradation and excretion of this molecule.

In connection with the present invention, there will be understood by “wound healing” the physiological processes for regeneration of destroyed tissue and for closing a wound, especially formation of new connective tissue and capillaries. Wound healing may be primary wound healing (first intention healing), which in the case of a clean wound is characterized by rapid and complication-free closure and largely complete recovery, resulting from minimal formation of new connective tissue between the wound edges, which have a good blood supply and have been approximated if necessary. In the case of wounds with wound edges that are further apart, especially crushed or necrotic wound edges, and of wound infections, delayed secondary wound healing (second intention healing) takes place. In such cases the tissue defect becomes filled with granulation tissue as a result of (a)bacterial inflammation, and scar tissue is formed more extensively. Epithelialization starting from the edge represents the completion of wound healing. Such wound healing is divided into three phases, known as latency phase, proliferative phase and repair phase. The latency phase in turn is divided into the exudative phase with scab formation, especially in the first few hours after the wound occurred, and the absorptive phase with catabolic autolysis, extending over a period of one to three days after the wound occurred. The proliferative phase is characterized by anabolic repair with production of collagen by fibroblasts, and it takes place on the fourth to seventh day after the wound occurred. The repair phase begins on the eighth day after the wound occurred, and is characterized by transformation of the granulation tissue into a scar.

In connection with the present invention, there will be understood by a “wound” a break in the continuity of body tissues with or without loss of substance, caused by mechanical injury or physically related cell damage. Within the meaning of the present invention, a wound is also considered to be a disease. Types of wound are mechanical wounds, thermal wounds, chemical wounds, radiation-related wounds and disease-related wounds. Mechanical wounds are caused by external violence and occur in particular as cut and stab wounds, crushing, lacerating, tearing and abrading wounds, scratch and bite wounds and projectile wounds. Thermal wounds are caused by exposure to heat or cold. Chemical wounds are caused in particular by burning with acids or alkalis. Radiation-related wounds are caused, for example, by exposure to actinic and ionizing radiation. Wounds occurring in relation to disease are in particular congestion-related wounds, traumatic wounds, diabetic wounds etc. According to the invention, it is provided in particular that low-dosage erythropoietin will be administered for wound healing, preferably topically or intravenously.

The present invention relates to the use of low-dosage erythropoietin for the therapy of hypercholesterolemia, diabetes mellitus, insulin resistance, endothelium-mediated chronic inflammatory disorders, endotheliosis including reticuloendotheliosis, atherosclerosis, age-related cardiovascular disorders, ischemic disorders of the extremities, preeclampsia, Raynaud's disease, hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, pregnancy-induced hypertension, chronic or acute renal failure, especially terminal renal failure, renal function restrictions with glomerular filtration rates of <80 ml/min, especially 30 to 80 ml/min, preferably 40 to 80 m/min, microalbuminuria, proteinuria, elevated ADMA levels or wounds and sequelae thereof.

According to the invention, it is provided that erythropoietin will be administered to a patient in a therapeutically effective dose sufficient to cure the condition of an aforementioned disease, especially a disease associated with a dysfunction of endothelial progenitor cells, or to prevent this condition, to stop the progression of such a disease and/or to alleviate the symptoms of such a disease. The dose to be administered to a patient depends on many factors, for example the age, body weight and sex of the patient, the severity of the disorders, etc.

“Inventive Dosing of EPO”

According to the invention, it is preferred for all uses, methods and compositions of the present teaching that erythropoietin be used in small quantities, smaller than the quantities known to be used for the treatment of renal anemia. Within the meaning of the present teaching, there will be understood by a small or low dose or dosage, especially in vivo, or in other words per patient, EPO doses of 1 to 2000, preferably 20 to 2000 units (IU; international units)/week, preferably doses of 20 to 1500 IU/week, especially doses of 20 to 1000 IU/week, especially doses of 20 to 950 IU/week, especially doses of 20 to 900 IU/week, especially doses of 20 to 850 IU/week, especially doses of 20 to 800 IU/week, especially doses of 20 to 750 IU/week, especially doses of 20 to 700 IU/week, especially doses of 20 to 650 IU/week, especially doses of 20 to 600 IU/week, especially doses of 20 to 550 IU/week, especially doses of 20 to 500 IU/week, especially doses of 20 to 450 IU/week, especially doses of 20 to 400 IU/week, especially doses of 20 to 350 IU/week, especially doses of 20 to 300 IU/week, especially doses of 20 to 250 IU/week, especially doses of 20 to 200 IU/week, especially doses of 20 to 150 IU/week, according to the severity of the disorder and depending on renal function. According to the invention, it is also provided that doses of 1 to 450, preferably 1 to 9 IU/week will be used. All of the foregoing doses provided according to the invention, for example of 1 to 2000 units (IU)/week per patient, especially, for example, of 500 to 2000 IU/week per patient, are subpolycythemic doses, or in other words doses that do not lead to an increase of the hematocrit, and in particular do not lead to an increase of more than 10%, especially 5%, preferably 2% in the hematocrit compared with the hematocrit prior to the treatment with EPO. The subpolycythemic doses provided according to the invention correspond to weekly doses of about 1 to 90 units (IU) of EPO/kg of body weight, especially 1 to 45, especially 1 to 30 IU of EPO/kg of body weight, especially 1 to 20 IU of EPO/kg of body weight, especially 1 to 15 IU of EPO/kg of body weight, especially 1 to 10 IU of EPO/kg of body weight, especially 1 to 4 IU of EPO/kg of body weight, or a comparable weekly dose of Aranesp of 0.001 to 0.4 μg/kg of body weight, 0.001 to 0.3 μg/kg of body weight, 0.001 to 0.25 μg/kg of body weight, 0.001 to 0.2 μg/kg of body weight, 0.001 to 0.15 μg/kg of body weight, 0.001 to 0.1 μg/kg of body weight, 0.001 to 0.09 μg/kg of body weight, 0.001 to 0.08 μg/kg of body weight, 0.001 to 0.07 μg/kg of body weight, 0.001 to 0.06 μg/kg of body weight, 0.001 to 0.05 μg/kg of body weight, 0.001 to 0.04 μg/kg of body weight, 0.001 to 0.03 μg/kg of body weight, 0.001 to 0.02 μg/kg of body weight, 0.001 to 0.01 μg/kg of body weight, 0.001 to 0.009 μg/kg of body weight, 0.001 to 0.008 μg/kg of body weight, 0.001 to 0.007 μg/kg of body weight, 0.001 to 0.006 μg/kg of body weight, 0.001 to 0.005 μg/kg of body weight, 0.001 to 0.004 μg/kg of body weight, 0.001 to 0.003 μg/kg of body weight, 0.001 to 0.002 μg/kg of body weight. Aranesp is a doubly PEGylated EPO.

According to the invention, it is particularly preferred for all uses, methods and compositions of the present teaching that erythropoietin be used in small quantities, smaller than the quantities known to be used for the treatment of renal anemia. Within the meaning of the present teaching, there will be understood by a small or low dose or dosage, especially in vivo, or in other words per patient, EPO doses of 0.001 to 90, preferably 0.001 to 50 units (IU; international units) per kilogram of body weight per week, especially doses of 0.05 to 45 IU/kg/week, especially doses of 0.05 to 40 IU/kg/week, especially doses of 0.05 to 35 IU/kg/week, especially doses of 0.05 to 33 IU/kg/week, especially doses of 0.05 to 31 IU/kg/week, especially doses of 0.05 to 29 IU/kg/week, especially doses of 0.05 to 27 IU/kg/week, especially doses of 0.05 to 25 IU/kg/week, especially doses of 0.05 to 23 IU/kg/week, especially doses of 0.05 to 21 IU/kg/week, especially doses of 0.05 to 20 IU/kg/week, especially doses of 0.05 to 19 IU/kg/week, especially doses of 0.05 to 17 IU/kg/week, especially doses of 0.05 to 15 IU/kg/week, especially doses of 0.05 to 13 IU/kg/week, especially doses of 0.05 to 11 IU/kg/week, especially doses of 0.05 to 9 IU/kg/week, especially doses of 0.05 to 7 IU/kg/week, especially doses of 0.05 to 5 IU/kg/week, especially doses of 0.05 to 3 IU/kg/week, especially doses of 0.05 to 1 IU/kg/week, according to the severity of the disorder and depending on renal function. According to the invention, it is also provided that doses of 0.001 to 20, preferably 0.05 to 10 IU/kg/week will be used. All of the foregoing doses provided according to the invention, for example of 0.01 to 90 units (IU)/kg/week per patient, especially, for example, of 0.01 to 50 IU/kg/week per patient, are subpolycythemic doses, or in other words doses that do not lead to an increase of the hematocrit, and in particular do not lead to an increase of more than 10%, especially 5%, preferably 2% in the hematocrit compared with the hematocrit prior to the treatment with EPO. The subpolycythemic doses provided according to the invention correspond to weekly doses of about 0.001 to 90 units (IU) of EPO/kg of body weight, especially 0.001 to 50, especially 0.001 to 45 IU of EPO/kg of body weight, especially 1 to 15 IU of EPO/kg of body weight, especially 1 to 10 IU of EPO/kg of body weight, especially 1 to 4 IU of EPO/kg of body weight, or a comparable weekly dose of Aranesp of 0.000005 to 0.45 μg per kilogram of body weight, 0.00025 to 0.250 μg/kg of body weight, 0.00025 to 0.225 μg/kg of body weight, 0.00025 to 0.2 μg/kg of body weight, 0.00025 to 0.175 μg/kg of body weight, 0.00025 to 0.165 μg/kg of body weight, 0.00025 to 0.155 μg/kg of body weight, 0.00025 to 0.145 μg/kg of body weight, 0.00025 to 0.135 μg/kg of body weight, 0.00025 to 0.125 μg/kg of body weight, 0.00025 to 0.115 μg/kg of body weight, 0.00025 to 0.105 μg/kg of body weight, 0.00025 to 0.095 μg/kg of body weight, 0.00025 to 0.085 μg/kg of body weight, 0.00025 to 0.075 μg/kg of body weight, 0.00025 to 0.065 μg/kg of body weight, 0.00025 to 0.055 μg/kg of body weight, 0.00025 to 0.045 μg/kg of body weight, 0.00025 to 0.035 μg/kg of body weight, 0.00025 to 0.025 μg/kg of body weight, 0.00025 to 0.015 μg/kg of body weight, 0.00025 to 0.005 μg/kg of body weight. Aranesp is a doubly PEGylated EPO. Compared with the initial dose of 90 to 150 IU/kg of body weight per week (beginning with 4000 to 8000 IU/week as a rule, but even much higher if the result of therapy is not satisfactory) usually used for therapy of renal anemia, the small doses cited above—for example the dose of 0.001 to 90 units/kg/week per patient, and especially, for example, of 0.001 to 50 units/kg/week per patient, as provided according to the invention for the treatment of diseases or pathological states associated with dysfunction of endothelial progenitor cells—are extremely low.

Unless otherwise specified, the cited dosages are one-time doses to be administered weekly, although they can also be divided into several individual doses in a week, or in other words administered by multiple dosing.

A particularly preferred embodiment of the invention relates to the use of low-dosage erythropoietin and/or its derivatives as defined in the foregoing section entitled “Inventive dosing of EPO” as active ingredient for production of a pharmaceutical composition or as a drug for the therapy of pathological conditions or diseases associated with a dysfunction of endothelial progenitor cells.

According to the invention, there will be understood by “active ingredient” an endogenous or exogenous substance that, on contact with target molecules or target cells or target tissues, influences specific functions of tissues, organs or organisms in differentiated manner. According to the invention, therefore, it is provided that erythropoietin, as active ingredient of the inventive pharmaceutical composition, upon contact with endothelial progenitor cells, will influence the proliferation, differentiation and/or migration behavior thereof in a human or animal organism in such a way that dysfunctions of endothelial progenitor cells can be compensated and the diseases occurring as a consequence of these dysfunctions can be effectively controlled, alleviated or eliminated, or these diseases can be effectively prevented. It is also provided that the use of low-dosage erythropoietin will lead both to organ regeneration and to slowing of the progression of functional restrictions in different organs and organ systems.

In connection with the present invention, there will be understood by “pharmaceutical composition” or “drug” a mixture used for diagnostic, therapeutic and/or preventive purposes, or in other words a mixture that promotes or restores the health of a human or animal body, which mixture includes at least one natural or synthetically produced active ingredient that brings about the therapeutic effect. The pharmaceutical composition may be either a solid or a liquid mixture. For example, a pharmaceutical composition that includes the active ingredient may contain one or more pharmaceutically tolerable components. The pharmaceutical composition may additionally include additives normally used in the art, for example stabilizers, finishing agents, release agents, disintegrants, emulsifiers or other substances normally used for production of pharmaceutical compositions.

According to the invention, there is provided in particular the use of erythropoietin, preferably in low doses, and/or a derivative thereof as active ingredient for producing a drug for the therapy of hypercholesterolemia, diabetes mellitus, insulin resistance, endothelium-mediated chronic inflammatory disorders such as vascular inflammations, endotheliosis including reticuloendotheliosis, atherosclerosis, age-related cardiovascular disease, ischemic disorders of the extremities, Raynaud's disease, hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, preeclampsia, pregnancy-induced hypertension, chronic or acute renal failure, especially terminal renal failure, renal function restrictions with glomerular filtration rates of <80 ml/min. especially 30, preferably 40 to 80 ml/min, microalbuminuria, proteinuria, elevated ADMA levels or wounds and sequelae thereof.

The inventive pharmaceutical composition may be suitable both for topical and for systemic administration.

In a preferred embodiment of the invention, it is provided that the pharmaceutical composition will be used for parenteral, especially intravenous, intramuscular, intracutaneous or subcutaneous administration. Preferably the erythropoietin-containing drug has the form of an injection or infusion.

In a further use, it is provided that the erythropoietin-containing pharmaceutical composition will be administered orally. For example, the erythropoietin-containing drug is administered in a liquid presentation such as a solution, suspension or emulsion, or a solid presentation such as a tablet.

In a further use, it is provided that the pharmaceutical composition will be suitable for pulmonary administration or for inhalation. According to the invention, therefore, it is provided that erythropoietin will be administered in therapeutically effective manner directly to the lungs of the patient. This form of administration of erythropoietin permits rapid delivery of an erythropoietin dose to a patient without the need to perform an injection. By absorption of erythropoietin through the lungs, considerable quantities of erythropoietin can be delivered via the lungs to the bloodstream, leading to elevated erythropoietin concentrations in the bloodstream. In a preferred embodiment of the invention, the pharmaceutical composition to be absorbed through the lungs is an aqueous or nonaqueous solution or a dry powder. When the erythropoietin-containing drug to be administered by the pulmonary route is in the form of a dry powder, the said powder preferably comprises erythropoietin-containing particles, wherein the particles have a diameter of smaller than 10 μm, thus enabling the drug to reach even distal regions of the patient's lungs. In a particularly preferred embodiment of the invention, it is provided that the drug to be administered by the pulmonary route will be in the form of an aerosol.

A particularly preferred embodiment of the invention relates to the use of erythropoietin for production of a pharmaceutical composition for therapy of diseases associated with a dysfunction of endothelial progenitor cells, wherein the pharmaceutical composition contains not only erythropoietin as active ingredient but also at least one further additional active ingredient for stimulation of endothelial progenitor cells.

The further active ingredient is preferably an active ingredient that in particular stimulates the physiological mobilization of endothelial progenitor cells from bone marrow or “other stem cell” niches. According to the invention, however, the further active ingredient may also be an active ingredient that in particular stimulates the dividing behavior, or in other words the proliferation, of endothelial progenitor cells. According to the invention, however, the possibility also exists that the further active ingredient will stimulate in particular the differentiation behavior and/or the migration behavior of endothelial progenitor cells. Particularly preferably, the further active ingredient that stimulates endothelial progenitor cells is VEGF, PIGF, GM-CSF, an HMG-CoA reductase inhibitor, especially a statin such as simvastatin, mevastatin or atorvastatin, an ACE inhibitor such as enalapril, ramipril or trandolapril, an AT-1 blocker such as irbesartan, lorsartan or olmesaratan, and/or an NO donor, especially L-arginine.

According to the invention, it is also provided that the at least one further active ingredient stimulates in particular differentiated endothelial cells, or in other words the proliferation and/or migration thereof, but not endothelial progenitor cells. Particularly preferably, it will be bFGF (basic fibroblast growth factor) or angiogenin.

A further embodiment of the invention relates to the use of erythropoietin and/or derivatives thereof as active ingredient for production of a pharmaceutical composition for stimulation of endothelial progenitor cells, especially for stimulation of mobilization, proliferation, differentiation to endothelial cells and/or migration toward a vasculogenic or angiogenic stimulus. According to the invention, it is further provided that erythropoietin and/or its derivatives will be used as active ingredient for production of a pharmaceutical composition for stimulation of vasculogenesis and/or endothelium formation, especially in the adult human or animal organism.

The present invention therefore also relates to pharmaceutical compositions for stimulation of endothelial progenitor cells, especially for stimulation of mobilization, proliferation, differentiation thereof to endothelial cells and/or migration toward a vasculogenic or angiogenic stimulus, for stimulation of vasculogenesis and/or endothelium formation and for treatment of diseases of the human or animal body that are associated with a dysfunction of endothelial progenitor cells and/or endothelial cells. In particular, the present invention relates to pharmaceutical compositions or drugs that contain erythropoietin as active ingredient and at least one further active ingredient for stimulation of endothelial progenitor cells and/or differentiated endothelial cells. In a preferred embodiment, the present invention relates to pharmaceutical compositions containing erythropoietin and at least one further active ingredient selected from the group comprising VEGF, PIGF, GM-CSF, an HMG-CoA reductase inhibitor, especially a statin such as simvastatin, mevastatin or atorvastatin, an ACE inhibitor such as enalapril, ramipril or trandolapril, an AT-1 blocker such as irbesartan, lorsartan or olmesaratan, an NO donor, especially L-arginine, bFGF and angiogenin.

A further preferred embodiment of the invention relates to the use of erythropoietin for production of a transplantable endothelial cell preparation. According to the invention, it is provided in particular in this embodiment that endothelial cells will be produced in vitro by cultivation of endothelial progenitor cells in the presence of erythropoietin and will then be transplanted into a recipient organism, especially an organism suffering from a disease associated with a dysfunction of endothelial progenitor cells. For example, mononuclear cells (MNC) can be isolated from blood by density gradient centrifugation and cultivated in suitable culture media in vitro. Methods for isolation and in vitro cultivation of mononuclear cells are described, for example, in Asahara, Science, 275 (1997), 964-967; Dimmeler et al., J. Clin. Invest., 108 (2001), 391-397 and Llevadot et al., J. Clin. Invest., 108 (2001) 399-405. The mononuclear cells are then further cultivated in the presence of erythropoietin, in order to stimulate the proliferation and differentiation behavior of the endothelial progenitor cells contained in the MNCs, and especially to increase the number of differentiated adherent endothelial cells. According to the invention, it is also provided that the MNCs will be cultivated in the presence of erythropoietin and at least one further substance that stimulates the proliferation and differentiation of endothelial progenitor cells. Particularly preferably, there is used as the further substance VEGF, PIGF, GM-CSF, an NO donor such as L-arginine, an ACE inhibitor such as enalapril, ramipril or trandolapril, an AT-1 blocker such as irbesartan, lorsartan or olmesaratan, or an HMG-CoA reductase inhibitor such as a statin, in particular simvastatin, mevastatin or atorvastatin.

In a further preferred embodiment of the invention, endothelial progenitor cells are applied to corresponding patients simultaneously with other cell populations usable for cell therapy, such as hepatocytes, myocytes, cardiomyocytes or island cells, after prior incubation with low-dosage erythropoietin in vitro and/or local as well as systemic application of low-dosage erythropoietin in vivo, in order in this way to ensure that the tissue cells usable for cell therapy settle with sufficient binding to the vascular system.

A further preferred embodiment of the invention also relates to the use of erythropoietin for production of a pharmaceutical composition or of a kit for sequential, timed successive or simultaneous administration of low-dosage erythropoietin as well as one or more other chemical, thermal, mechanical or biological agents, in order to mobilize certain sites of a patient's body, such as implantation target sites, in order to increase the number and function of endothelial progenitor cells and/or to bring about regeneration or slowing of the progression of tissue damage. Such mechanical agents can be, for example, endoprostheses, preferably implantation supports for teeth, bones or ligament/tendon replacements. Furthermore, the biological agents can be solid organs such as liver, kidneys, heart, pancreas or skin, or even hair implants. The invention therefore provides that EPO, especially in low doses, will be used so that mechanical agents such as endoprostheses or biological agents implanted simultaneously, subsequently or beforehand can grow or be integrated better, faster and more efficiently into the surrounding body structure. The invention therefore also relates to the use of erythropoietin for production of a pharmaceutical composition or of a kit for improving, especially for promoting and/or accelerating, integration of biological agents or endoprostheses into surrounding body structures, especially of teeth, tooth replacements, tooth implants or other endoprostheses, such as bone replacements, bone implants, especially hip joint prostheses or ligament/tendon replacements, such as cruciate ligaments. In this connection, it can be provided if necessary that the erythropoietin will be used together with cell populations suitable for cell therapy and/or endothelial progenitor cells. In the aforesaid use of erythropoietin for production of a pharmaceutical composition or of a kit for improving, especially for promoting and/or accelerating, integration of biological or mechanical agents into target structures, especially target tissue, target bones or target cartilage of a patient, it can be provided in a further preferred embodiment that the mechanical agents to be used are made, for example, of steel, ceramic, plastic or another material. In addition, it can be provided that osteoblasts, cells having osteogenic potential, thrombocytes, blood cells or similar agents can be used in the present application as cell populations suitable for cell therapy. In a further preferred embodiment, it can be provided that the mechanical agent in particular to be used be contained in the pharmaceutical composition or in the pharmaceutical kit together with organic adhesive, such as a fibrin glue.

In a further preferred embodiment of the invention, there is provided the use of erythropoietin or suitable active ingredients for topical application in the sense of “beauty care”, especially for prevention or timely reduction of creases and wrinkles, strengthening of the connective tissue, protection and tightening of the skin, especially facial skin, against harmful environmental factors, and as makeup foundation. The topical application of erythropoietin is intended to counteract the formation and further development of age spots, to refine the skin texture and to support not only the skin rejuvenation process, preferably by accelerated reepithelialization, but also hair growth.

In a further form of application, there is provided the administration of low-dosage erythropoietin in a manner adapted to its circadian rhythm. Endogenous erythropoietin production has its acro phase (daily maximum) in the late afternoon, and so the administration of the low-dosage erythropoietin should preferably take place in the morning, especially between 6:00 and 10:00 a.m., in order in this way to achieve a maximum biological effect.

In a further embodiment of the invention, there is provided the use of low-dosage erythropoietin for pretreatment and/or further treatment of tissues or organs to be transplanted. In this case, the transplants are treated with low-dosage erythropoietin before transplantation, preferably immediately before, while still in the donor organism. The recipient organism can also be treated with low-dosage erythropoietin from the time of transplantation onward. By this erythropoietin treatment of the organs or tissues to be transplanted, both directly before and after transplantation, it is ensured according to the invention that new blood vessels will form rapidly by induced vasculogenesis in the transplant after transplantation has taken place into a body, and that these newly formed blood vessels will be rapidly connected to the blood system of the recipient organism. The formation of endothelia is also achieved rapidly in this way. Such treatment of organ or tissue transplants with low-dosage erythropoietin therefore achieves faster growth of these systems into the body, whereby the risk of rejection is considerably reduced. Furthermore, organ regeneration is stimulated by the administration of low-dosage erythropoietin.

In a further embodiment of the invention, it is provided that the organ or tissue transplants will be treated before transplantation with low-dosage erythropoietin in combination with at least one further factor that stimulates endothelial progenitor cells. This factor is preferably a substance selected from the group comprising VEGF, PIGF, GM-CSF, an HMG-CoA reductase inhibitor, for example a statin, especially simvastatin, mevastatin or atorvastatin, an ACE inhibitor such as enalapril, ramipril or trandolapril, an AT-1 blocker such as irbesartan, lorsartan or olmesaratan or an NO donor, especially L-arginine. In a further embodiment, it is provided that the organ or tissue transplants will be treated before transplantation not only with erythropoietin but also with a further substance that stimulates proliferation and migration of differentiated endothelial cells. Particularly preferably, this substance is angiogenin or bFGF. In a further embodiment, it is provided that the pretreatment of the organ or tissue transplants with erythropoietin will take place using isolated endothelial progenitor cells, which have been expanded in vitro if necessary.

In a further particularly preferred embodiment of the invention, it is provided that low-dosage erythropoietin will be used to produce implantable or transplantable, cell-containing, in-vitro organs or tissues. According to the invention, it is provided in particular that the organ or tissue produced in vitro will be treated prior to transplantation or implantation with low-dosage erythropoietin in vitro, in order to stimulate the endothelial progenitor cells present in the body of the recipient organism, and especially physiological mobilization, migration, proliferation and differentiation thereof. After transplantation or implantation of the in-vitro organ or tissue, the recipient organism is preferably further treated with low-dosage erythropoietin in the inventive doses. By treatment of the in-vitro organ or tissue with erythropoietin prior to transplantation or implantation, and by post-treatment of the recipient organism with erythropoietin if necessary, it is ensured according to the invention that new blood vessels will form rapidly by induced vasculogenesis in the in-vitro organ or tissue system after transplantation or implantation has taken place into a body, and that these newly formed blood vessels will be rapidly connected to the blood system of the recipient organism. Rapid formation of endothelia and thus reendothelialization is also achieved in this way. Such treatment of the in-vitro organ or tissue transplant systems with low-dosage erythropoietin therefore achieves faster growth of these systems into the body, whereby the risk of rejection is considerably reduced, and it also serves to protect the transplant.

By “in-vitro organ or tissue system” there will be understood a transplantable or implantable cell-containing tissue or organ, which is produced in vitro, under defined culture conditions, using defined cells and/or defined tissues. By “implantable in-vitro organ or tissue system” there will be understood a system that includes not only cells but also exogenous materials. By “transplantable in-vitro organ or tissue system” there will be understood in particular a cell-containing system that contains not only cells, tissue or organs of the same or of a different individual but also endogenous substances. In-vitro organs or tissues are characterized in particular by the fact that their structure corresponds largely to that of the native organs or tissues to be replaced, thus enabling them to assume the function of the replaced native organs or tissues in vivo.

In one inventive embodiment, it is provided that, prior to transplantation or implantation, the in-vitro organ or tissue systems will be treated with erythropoietin in combination with at least one further factor that stimulates endothelial progenitor cells. This factor is preferably one or more substances selected from the group comprising VEGF, PIGF, GM-CSF, an HMG-CoA reductase inhibitor, especially simvastatin, mevastatin or atorvastatin, an ACE inhibitor such as enalapril, ramipril or trandolapril, an AT-1 blocker such as irbesartan, lorsartan or olmesaratan, and an NO donor. In a further embodiment, it is provided that, prior to transplantation or implantation, the in-vitro organ or tissue systems will be treated not only with erythropoietin but also with a further substance that stimulates proliferation and migration of differentiated endothelial cells. Particularly preferably, this substance is angiogenin or bFGF. In a further embodiment, it is provided that the in-vitro organ or tissue systems will additionally contain isolated endothelial progenitor cells, which have been expanded in vitro if necessary.

A further preferred embodiment of the invention relates to the use of low-dosage erythropoietin for production of vascular prostheses or heart valves, wherein the vascular prostheses or heart valves are coated with erythropoietin prior to insertion into a body, especially a human body. By such coating of the vascular prostheses or heart valves with erythropoietin, it is ensured that endothelial progenitor cells in the body of the recipient organism will be stimulated. In particular, their mobilization from bone marrow, their proliferation, their differentiation to endothelial cells and their migration to the inserted vascular prostheses or heart valves will be stimulated. After the vascular prosthesis or heart valves produced in this way have been introduced into a body, such a body can be treated further with erythropoietin, especially in the inventive doses. Thereby endothelial layers form more rapidly on the inserted vascular prostheses, and growth into the relevant area of the body takes place more rapidly. In a preferred embodiment, it is provided that isolated endothelial progenitor cells, which have been expanded in vitro if necessary, are additionally used for coating the vascular prostheses and heart valves.

The present invention also relates to a method for stimulation of endothelial cell formation in vitro, comprising

a) isolation of cell populations containing endothelial progenitor cells from blood by means of density gradient centrifugation,

b) cultivation of the isolated cell populations comprising endothelial progenitor cells in cell culture medium, and

c) cultivation of the cell populations in the presence of low-dosage erythropoietin.

According to the invention, cultivation of the cell populations can take place in the presence of a further substance that stimulates endothelial progenitor cells.

The present invention further relates to a method for treatment of diseases associated with a dysfunction of endothelial progenitor cells, wherein erythropoietin, in a small dose such as explained in the section entitled “Inventive dosing of EPO”, alone or in combination with at least one other chemical, thermal, mechanical and biological agent, is administered to a patient with such a disease. The inventive method is suitable in particular for treating diseases of the human body such as hypercholesterolemia, diabetes mellitus, insulin resistance, endothelium-mediated chronic inflammatory disorders such as vascular inflammations, endotheliosis including reticuloendotheliosis, atherosclerosis, age-related cardiovascular disorder, ischemic disorders of the extremities, Raynaud's disease, hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, preeclampsia, pregnancy-induced hypertension, acute or chronic renal failure, especially terminal renal failure, renal function restrictions with glomerular filtration rates of <80 ml/min, especially 30 to 80 ml/min, preferably 40 to 80 ml/min, microalbuminuria, proteinuria, elevated ADMA levels or wounds and sequelae.

In a preferred embodiment of the inventive method for treatment of diseases associated with a dysfunction of endothelial progenitor cells, it is provided that there will be administered to the patient not only erythropoietin but also at least one further active ingredient selected from the group comprising VEGF, PIGF, GM-CSF, an HMG-CoA reductase inhibitor and an NO donor. Preferably the administered HMG-CoA reductase inhibitor will be a statin such as simvastatin, mevastatin or atorvastatin. The administered ACE inhibitor will be an active ingredient such as enalapril, ramipril or trandolapril. The administered AT-1 blocker will be active ingredients such as irbesartan, lorsartan or olmesaratan. The administered NO donor will preferably be L-arginine.

In a further preferred embodiment of the inventive method for treatment of diseases associated with a dysfunction of endothelial progenitor cells, it is provided that endothelial progenitor cells will be isolated from the blood of a human organism, expanded in vitro using low-dosage erythropoietin and differentiated to endothelial cells, after which the differentiated endothelial cells or the endothelial progenitor cells undergoing differentiation will be purified and isolated, then transplanted selectively into a patient's body region, tissue or organ that has been damaged because of the dysfunction of endothelial progenitor cells and/or endothelial cells, in order to induce local formation of new endothelium therein. In this way the damaged body regions, tissues and/or organs of the patient can be treated more selectively and rapidly. This embodiment of the inventive method for treatment of diseases associated with a dysfunction of endothelial progenitor cells comprises the following steps:

a) isolation of cell populations containing endothelial progenitor cells from blood by means of density gradient centrifugation,

b) cultivation of the cell populations containing endothelial progenitor cells in cell culture medium,

c) cultivation of the cell populations containing endothelial progenitor cells in the presence of low-dosage erythropoietin in order to stimulate proliferation of endothelial progenitor cells and/or differentiation thereof to endothelial cells,

d) isolation and purification of the differentiated endothelial cells, and

e) transplantation of the differentiated endothelial cells into a body with a disease associated with a dysfunction of endothelial progenitor cells.

After transplantation of the differentiated endothelial cells into a body, such a body can be treated further with erythropoietin, especially in the low doses provided according to the invention, or in other words the doses defined in the section entitled “Inventive dosing of EPO”, for example of 1 and preferably 0.001 to 90 IU/kg/week or of 20 to 2000 IU/week.

According to the invention, the cell populations containing endothelial progenitor cells can be cultivated in vitro in the presence of at least one further active ingredient selected from the group comprising VEGF, PIGF, GM-CSF, an HMG-CoA reductase inhibitor, an ACE inhibitor, an AT-1 blocker and an NO donor. Preferably the HMG-CoA reductase inhibitor used for cultivation will be a statin such as simvastatin, mevastatin or atorvastatin, the ACE inhibitors will be substances such as enalapril, ramipril or trandolapril, and the AT-1 blocker will be substances such as irbesartan, lorsartan or olmesaratan.

According to the invention, cell populations containing endothelial progenitor cells can be treated with sequential, timed successive or simultaneous administration of low-dosage erythropoietin as well as one or more other chemical, thermal, mechanical or biological agents, in order in this way to increase the number and function of endothelial progenitor cells and/or to bring about regeneration or slowing of the progression of tissue damage.

A further preferred embodiment of the invention relates to a method for treatment of vascular disorders, comprising:

a) isolation of cell populations containing endothelial progenitor cells from blood by means of density gradient centrifugation,

b) cultivation of the cell populations containing endothelial progenitor cells in cell culture medium,

c) cultivation of the cell populations containing endothelial progenitor cells in the presence of erythropoietin in order to stimulate proliferation of endothelial progenitor cells and/or differentiation thereof to endothelial cells,

d) isolation and purification of the differentiated endothelial cells, and

e) transplantation of the endothelial cells into a body with a vascular disorder.

After transplantation of the endothelial cells into the body with a vascular disorder, such a body can be further treated with erythropoietin, especially in the low doses according to the invention, or in other words the doses defined in the section entitled “Inventive dosing of EPO”, for example of 0.001 to 90 units/kg/week or of 20 IU/week to 2000 IU/week.

According to the invention, it is possible to cultivate the cell populations containing endothelial progenitor cells in the presence of at least one further active ingredient selected from the group comprising VEGF, PIGF, GM-CSF, an ACE inhibitor, an AT-1 blocker and/or an HMG-CoA reductase inhibitor. Preferably the ACE inhibitor used for cultivation will be substances such as enalapril, ramipril or trandolapril, and the AT-1 blocker used for cultivation will be substances such as irbesartan, lorsartan or olmesaratan, and the HMG-CoA reductase inhibitor used for cultivation will be a statin such as simvastatin, mevastatin or atorvastatin.

According to the invention, cell populations containing endothelial progenitor cells can be treated with sequential, timed successive or simultaneous administration of low-dosage erythropoietin as well as one or more other chemical, thermal, mechanical or biological agents, in order in this way to increase the number and function of endothelial progenitor cells and/or to bring about regeneration or slowing of the progression of tissue damage. Such mechanical agents can be endoprostheses, preferably implantation supports for teeth, bones or ligament/tendon replacements. Furthermore, the biological agents can be solid organs such as liver, kidneys, heart, pancreas or skin, or even hair implants. The invention therefore provides that EPO, especially in low doses, will be used so that mechanical agents such as endoprostheses or biological agents implanted simultaneously, subsequently or beforehand can grow or be integrated better, faster and more efficiently into the surrounding body structure. The invention therefore also relates to the use of erythropoietin for production of a pharmaceutical composition or of a kit for improving, especially for promoting and/or accelerating, integration of biological agents or endoprostheses into surrounding body structures, especially of teeth, tooth replacements, tooth implants or other endoprostheses, such as bone replacements, bone implants, especially hip joint prostheses or ligament/tendon replacements, such as cruciate ligaments. In a preferred embodiment, it can then be provided that the erythropoietin will be used together with cell populations suitable for cell therapy and/or endothelial progenitor cells. In the aforesaid use of erythropoietin for production of a pharmaceutical composition or of a kit for improving, especially for promoting and/or accelerating, integration of biological or mechanical agents into target structures, especially target tissue, target bones or target cartilage of a patient, it can be provided in a further preferred embodiment that the mechanical agents to be used are made, for example, of steel, ceramic, plastic or another material. In addition, it can be provided that osteoblasts, cells having osteogenic potential, thrombocytes, blood cells or similar agents can be used in the present application as cell populations suitable for cell therapy. In a further preferred embodiment, it can be provided that the mechanical agent in particular to be used will be contained in the pharmaceutical composition or in the pharmaceutical kit together with organic adhesive, such as a fibrin glue.

The inventive method for treatment of vascular disorders thus provides that endothelial progenitor cells will be isolated from the blood of a human organism, expanded in vitro using low-dosage erythropoietin and differentiated to endothelial cells, after which the differentiated endothelial cells or the endothelial progenitor cells undergoing differentiation will be purified and isolated, then transplanted selectively into a damaged blood vessel or an ischemic region, in order to induce local neovascularization therein. In this way damaged blood vessels or ischemic tissues can be treated more selectively and rapidly. The inventive method for treatment of vascular disorders is suitable in particular for treatment of vascular disorders such as ischemia, especially cerebral ischemia, ischemic disorders of the extremities, stroke, acute arterial occlusion, arterial occlusive disease, Raynaud's disease and ergotism.

Further advantageous embodiments of the invention are specified in the dependent claims.

The invention will be explained in more detail on the basis of the figures and examples hereinafter.

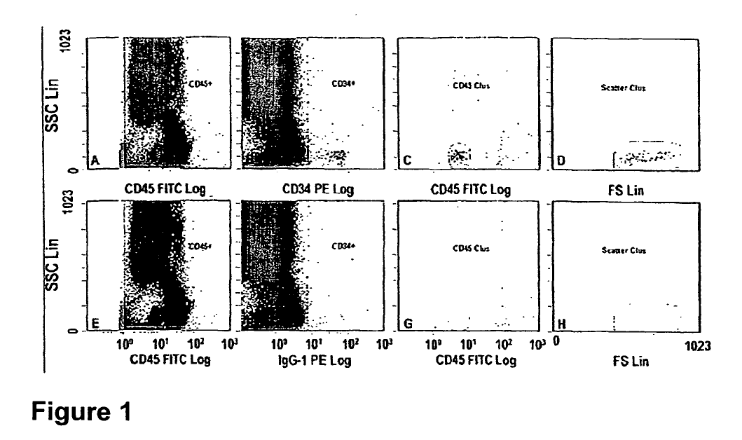

FIG. 1 shows the results of a FACS analysis of circulating CD34+ stem cells (cSC). (A-D): patients' samples; (E-F): isotype controls. cSC were identified by means of the additional expression of the CD34 marker (B and F), by means of the characteristic low to moderate CD45 antigen expression (C and G) and by means of the characteristic light scattering properties (D and H). The absolute cSC number was calculated per 100,000 monocytes and lymphocytes.

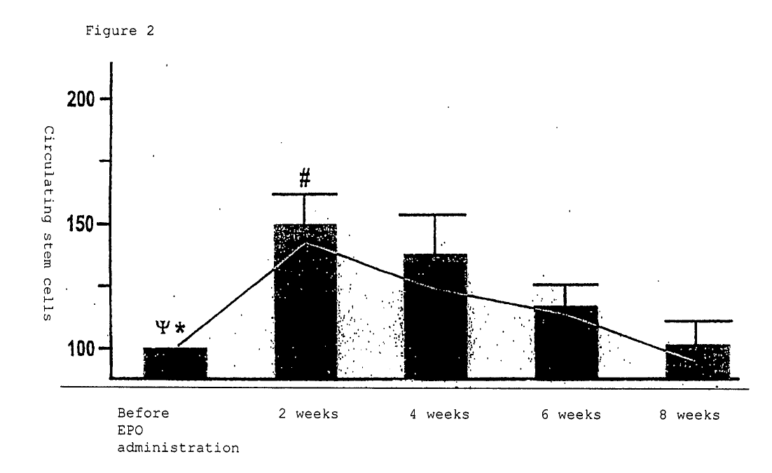

FIG. 2 shows a quantitative assay of circulating stem cells by means of flow cytometry. The figure shows the time-dependent effect of erythropoietin treatment using rhEPO (recombinant human erythropoietin) after 0, 2, 4, 6 and 8 weeks. n=11, the values correspond to mean values±standard deviation. Medians are shown by lines.

*: p<0.01 in the comparison at 2 weeks; ψ: p<0.05 in the comparison at 4 weeks, #: p<0.05 in the comparison at 8 weeks.

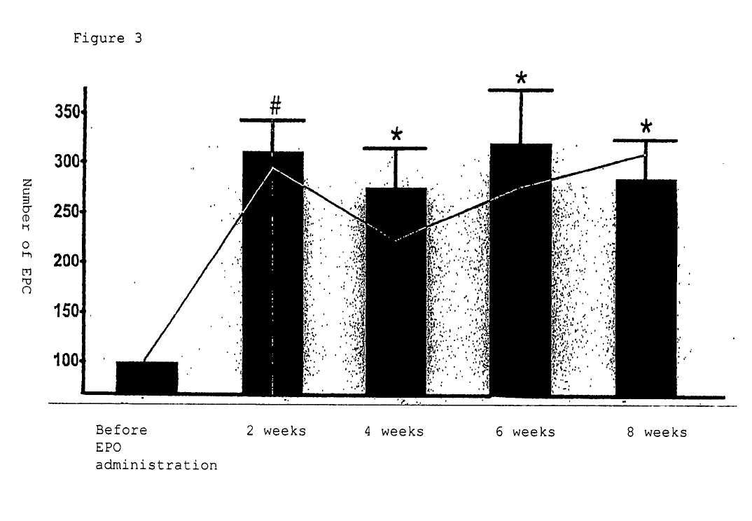

FIG. 3 shows a quantitative assay of cultivated endothelial progenitor cells (EPC). The figure shows that rhEPO treatment increases the relative number of EPCs. EPCs were isolated before the treatment of kidney patients with rhEPO and 2, 4, 6 and 8 weeks after treatment of the patients with rhEPO, and were characterized by means of their adhesion ability and the two markers acLDL-Dil and UEA-1 FITC. n=11, the values correspond to mean values±standard deviation. Medians are shown as lines.

*: p<0.01 compared with the period before treatment; #: p<0.001 compared with the period before treatment.

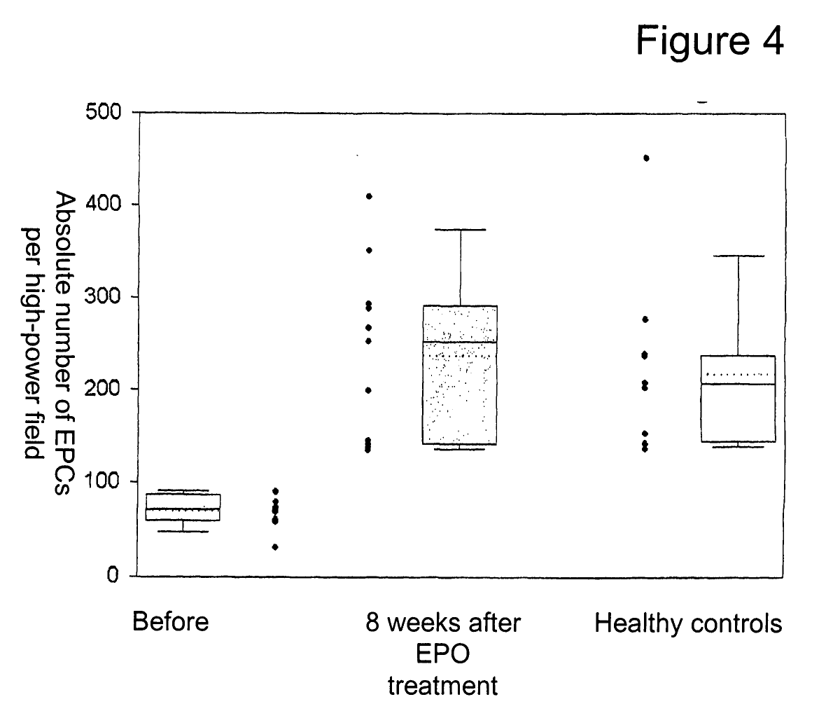

FIG. 4 shows the quantitative assay of cultivated endothelial progenitor cells (EPC). The figure shows that the absolute number of EPCs before initiation of rhEPO therapy is significantly reduced compared with healthy subjects of matched age and sex. Patients with renal anemia therefore exhibit distinct EPC dysfunction compared with control subjects. This reduced number of functional EPC was compensated 8 weeks after initiation of rhEPO therapy for renal anemia. EPCs were isolated before the treatment of kidney patients with rhEPO and 2, 4, 6 and 8 weeks after treatment of the patients with rhEPO, and were characterized by means of their adhesion ability and the two markers acLDL-Dil and UEA-1 FITC. n=11. The example shown is the course over 8 weeks and all the controls. The absolute values are shown on the one hand as individual values. In addition, box plots are presented (90th/75th/50th/25th and 10th percentiles as well as the mean value). Subjects of matched age and sex in whom EPCs were isolated and characterized analogously (n=11) served as healthy control.

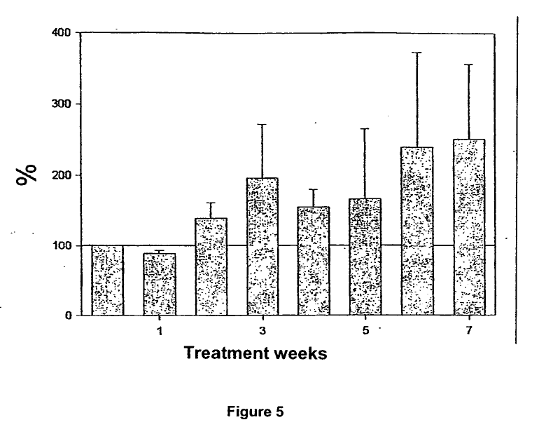

FIG. 5 shows the quantitative assay of cultivated endothelial progenitor cells (EPC) in healthy young subjects. The figure shows that treatment with rhEPO (30 IU of epoetin beta per kg of body weight per week) increases the relative number of EPCs. EPCs were isolated before the treatment of the subjects with rhEPO as well as weekly at 1, 2, 3, 4, 5, 6 and 7 weeks after treatment of the patients with rhEPO, and were characterized by means of their adhesion ability and the two markers acLDL-Dil and UEA-1 FITC. n=4, the values correspond to mean values±standard deviation.

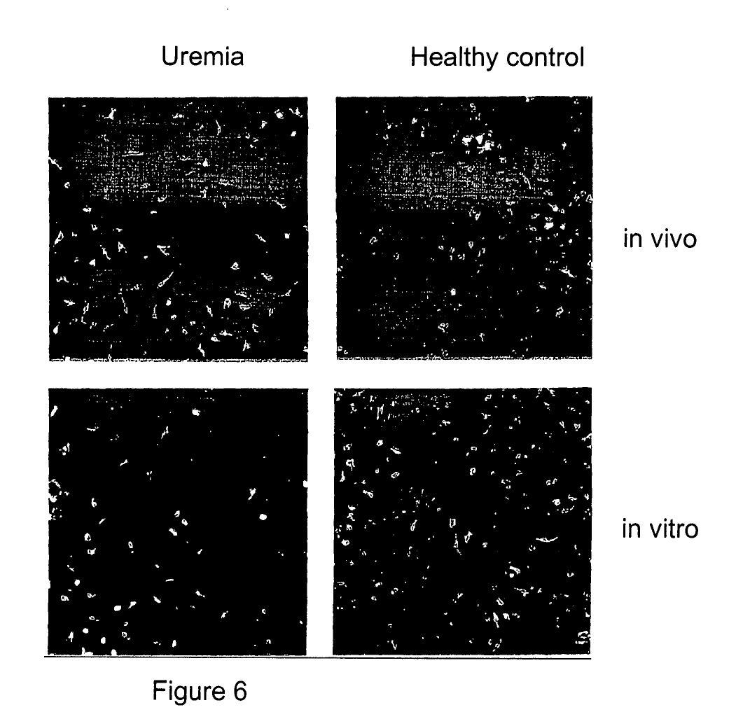

FIG. 6 shows a quantitative assay of cultivated endothelial progenitor cells (EPC). The representative photographs show that the absolute number of EPCs in uremic patients is significantly reduced compared with healthy subjects of matched age and sex (top row=in vivo). Patients with restricted renal function therefore exhibit distinct EPC dysfunction compared with control subjects. If endothelial progenitor cells of a healthy subject are cocultivated with serum of uremic patients, the differentiation ability of his or her endothelial progenitor cells is reduced (bottom row=in vitro). Thus restricted renal function with uremia derived therefrom leads to dysfunction of endothelial progenitor cells.

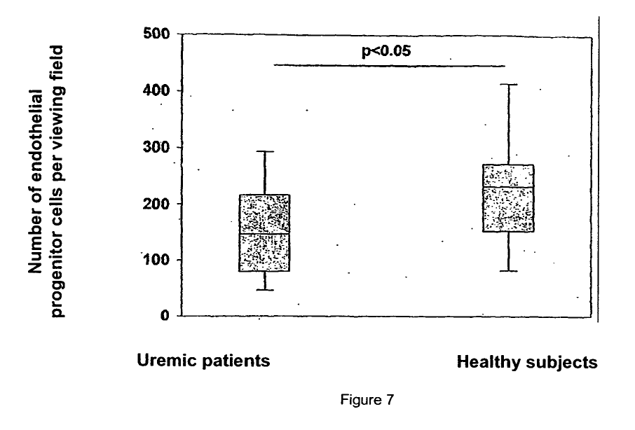

FIG. 7 shows a quantitative assay of cultivated endothelial progenitor cells (EPC) in 46 uremic patients with restricted renal function versus 46 subjects of matched age and sex, presented in the form of box plots (90th/75th/50th/25th and 10th percentiles as well as the mean value). The number of endothelial progenitor cells in the uremic patients is significantly reduced compared with the healthy subjects. Patients with restricted renal function therefore exhibit distinct EPC dysfunction compared with control subjects.

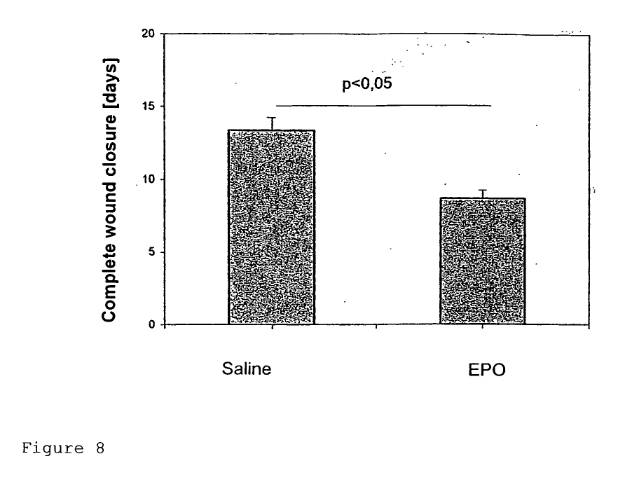

FIG. 8 shows the effect of erythropoietin on wound healing. The figure shows that, when a standardized skin wound inflicted on mice using a tissue punch was treated with erythropoietin, it already closed completely after seven to eight days. In contrast, when the wound was treated with physiological salt solution (saline), it did not close completely until after thirteen to fourteen days. Treatment with erythropoietin or physiological salt solution began 7 days before the skin wound was inflicted. Recombinant human erythropoietin was administered one time per week by s.c. (subcutaneous) injection (0.1 mg/kg Aranesp) (n=5 in each group).

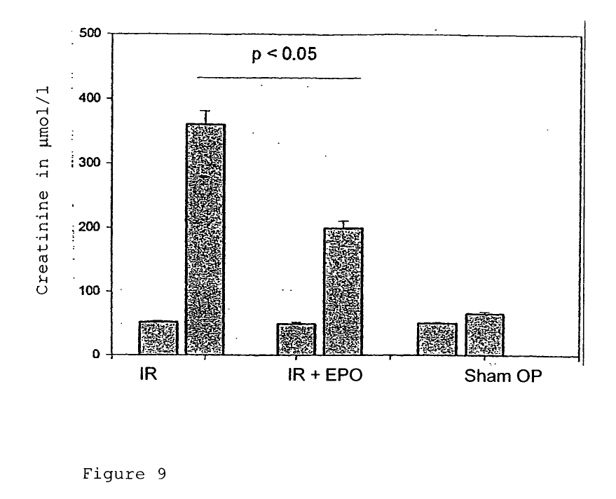

FIG. 9 shows that erythropoietin reduces the loss of renal function after acute renal failure (acute renal insufficiency). Sprague Dawley rats (250 to 300 g) were included in the study. The rats were anesthetized with ketamine (120 mg/kg) and Rompun (10 mg/kg). One of the experimental groups received 0.1 μg of Aranesp per kg of body weight one time on the day before induction of the acute renal failure. For comparison, there was used a group of experimental animals, each of which was given an s.c. injection of saline at the same time. By application of an arterial clamp to the right renal arteries, the blood flow into the kidney was interrupted for 45 minutes. During this period, a left nephrectomy was performed. A sham operation was performed on a further control group. In this procedure, the abdomen was opened to expose the left renal artery, but the blood supply was not interrupted and the contralateral right kidney was removed. All animals were anesthetized for 60 min and killed 24 h after the operation. In the animals treated with saline, the 45-minute ischemia with subsequent reperfusion of the remaining right kidney led to massive acute loss of renal function. This is reflected by the fact that the serum creatinine level 24 h after ischemia and reperfusion was 7 times higher than the level before ischemia and reperfusion (p<0.05). In contrast, the animals treated with the erythropoietin analog Aranesp exhibited only a four-fold increase in the serum creatinine levels one day after induction of damage by ischemia and reperfusion. No increase in retention levels was found in the animals subjected to left nephrectomy and a sham operation on the right kidney. The figure shows the creatinine concentration in the serum of EPO-treated animals (IR+EPO), NaCl-treated animals (IR) and sham-operated animals (sham OP) before ischemia-reperfusion (IR) injury and 24 hours thereafter. It is evident from the figure that the serum creatinine concentration 24 hours after ischemia-reperfusion injury is almost halved in the animals treated with Aranesp compared with the control without Aranesp (NaCl treatment).

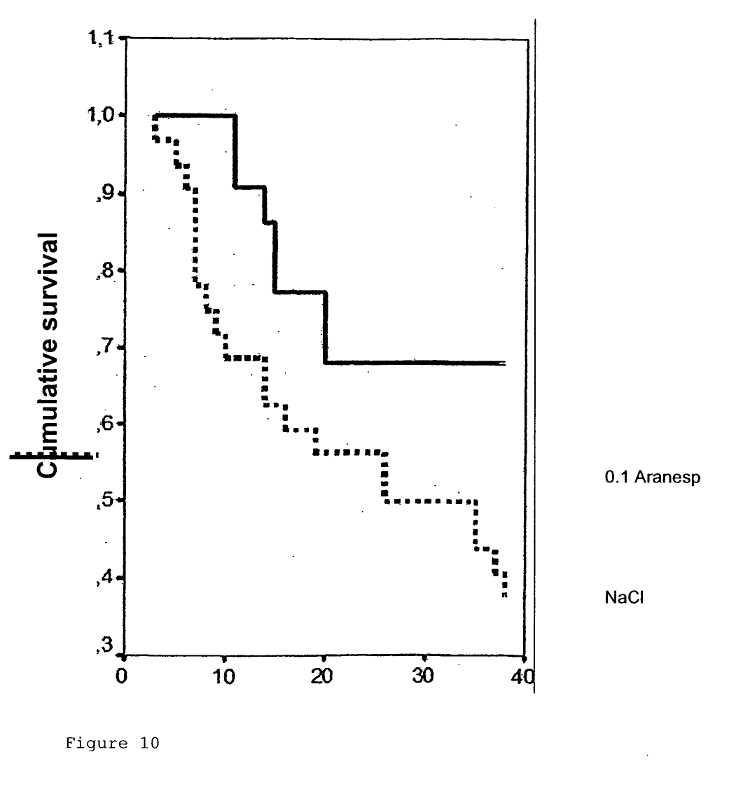

FIG. 10 shows the Kaplan-Mayer survival curves of two experimental groups treated either with Aranesp or NaCl after induction of chronic renal failure. 8-week old Sprague Dawley rats were included in the study. The rats were anesthetized with ketamine (120 mg/kg) and Rompun (10 mg/kg). Their right kidney was removed on day 0 and was immediately fixed in formalin for histological examination. The segmental arteries supplying the upper and lower renal poles of the left kidney were ligated. Thereby renal infarction occurred in the corresponding kidney areas, and only the middle third of the kidney remained functional. One time per week, the rats received Aranesp (0.1 μg/kg of body weight) or NaCl by s.c. injection. The animals treated with the erythropoietin analog Aranesp exhibited a significant survival advantage compared with the animals treated with saline (p=0.027; log rank test).

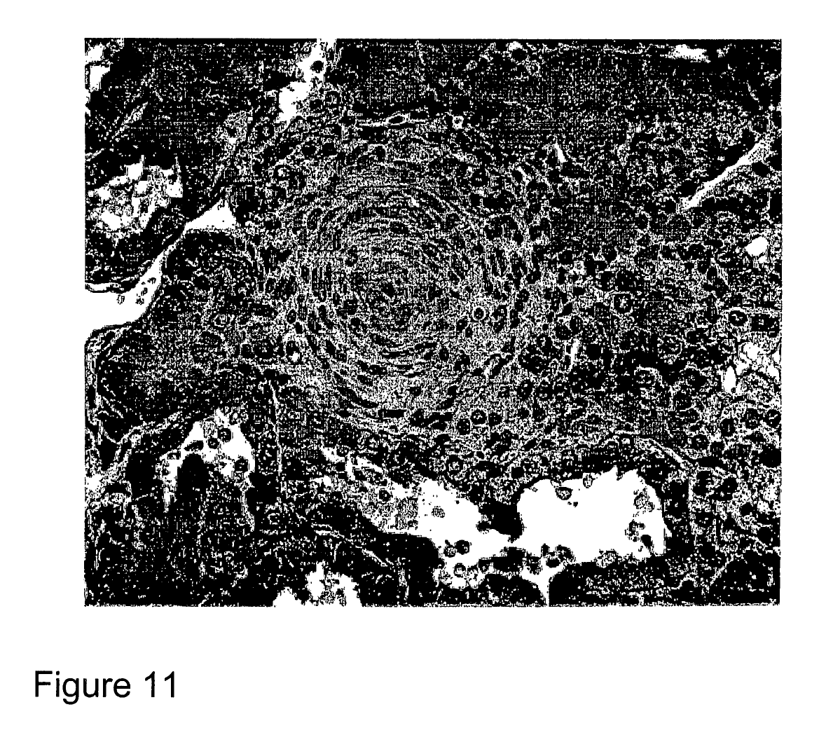

For the two experimental groups that were treated either with Aranesp or NaCl and whose Kaplan-Mayer survival curves are illustrated in FIG. 10, FIGS. 11 to 18 show optical microscopic kidney sections 6 weeks after induction of chronic renal failure.

FIG. 11 shows the histological changes in a Sprague-Dawley rat with chronic renal failure after NaCl treatment one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a medium-sized preglomerular artery with characteristic onionskin-like vessel wall proliferation associated with severe hypertensive damage, known as Fahr's malignant nephrosclerosis with endarteritis.

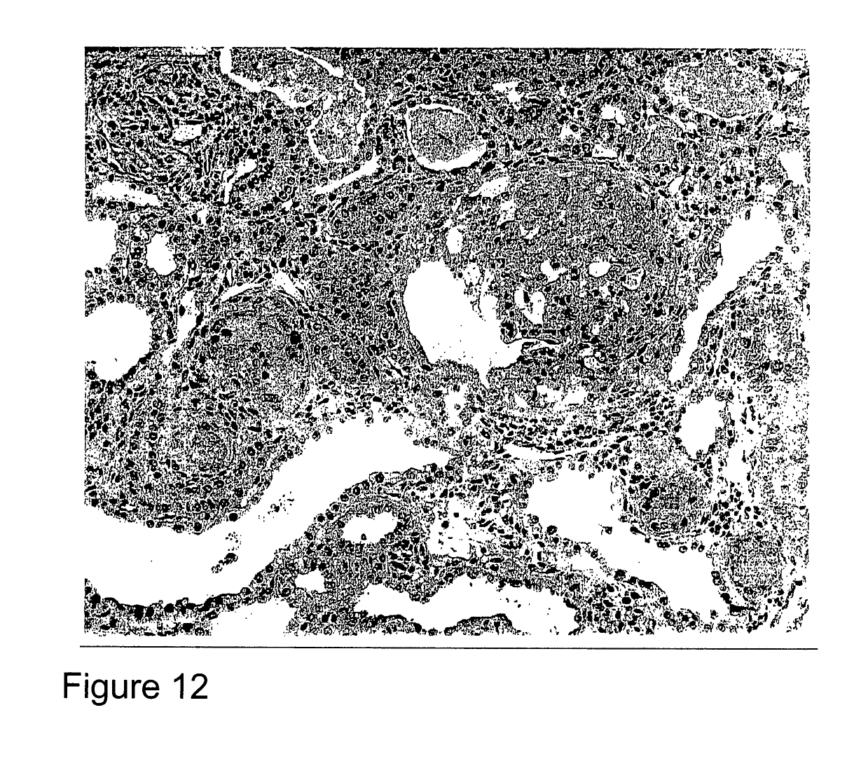

FIG. 12 shows the histological changes in a Sprague-Dawley rat with chronic renal failure after NaCl treatment one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows florid focal-segmental glomerulosclerosis, known as proliferative FSGS (right glomerulus). The other glomerulus (left) exhibits ischemic collapse of the loop convolution. A small vessel with severe endothelial damage is visible in the lower part of the photograph. The observed histological changes correspond to hypertensive organ damage or changes associated with overload nephropathy following 5/6 nephrectomy.

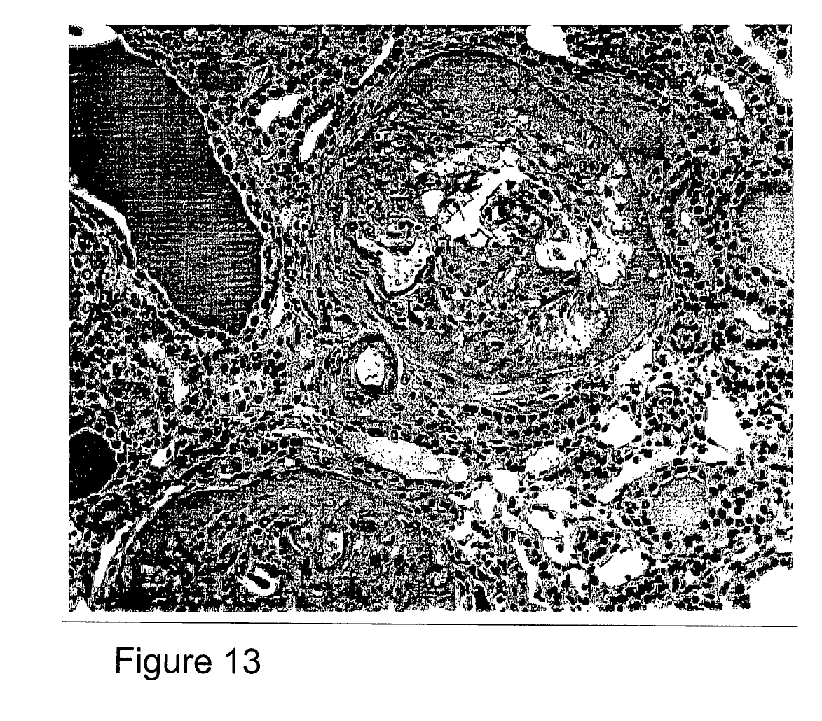

FIG. 13 shows the histological changes in a Sprague-Dawley rat with chronic renal failure after NaCl treatment one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows almost complete sclerosis or destruction of a glomerulus with compensatory enlargement and pronounced hyalinosis or fibrinoid necrosis of the associated afferent arterioles.

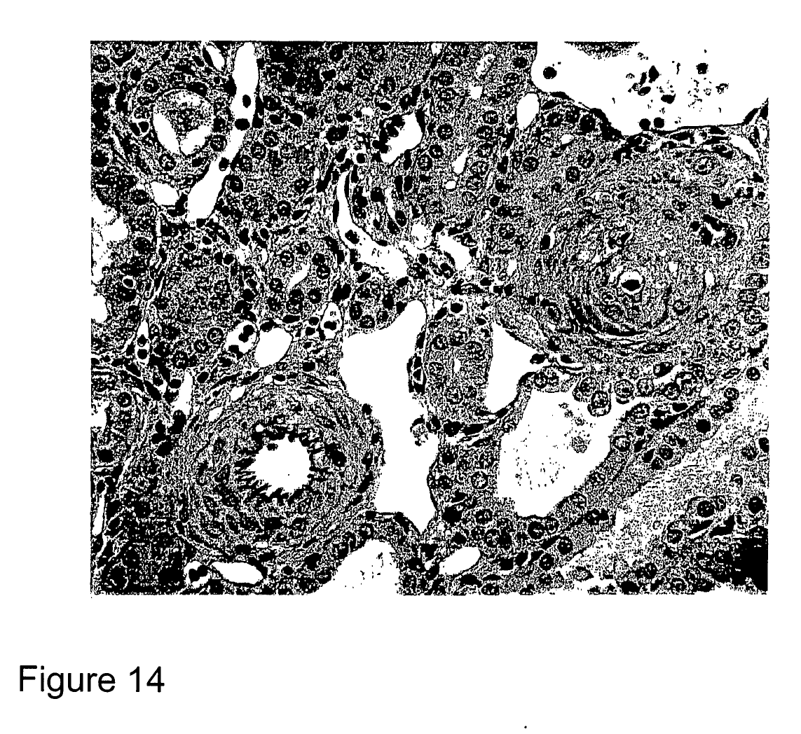

FIG. 14 shows the histological changes in a Sprague-Dawley rat with chronic renal failure after NaCl treatment one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a small preglomerular artery with characteristic onionskin-like vessel wall proliferation and wall necrosis associated with severe hypertensive damage, known as malignant nephrosclerosis (see right photograph). A normal (and as yet) undamaged arteriole is visible on the left.

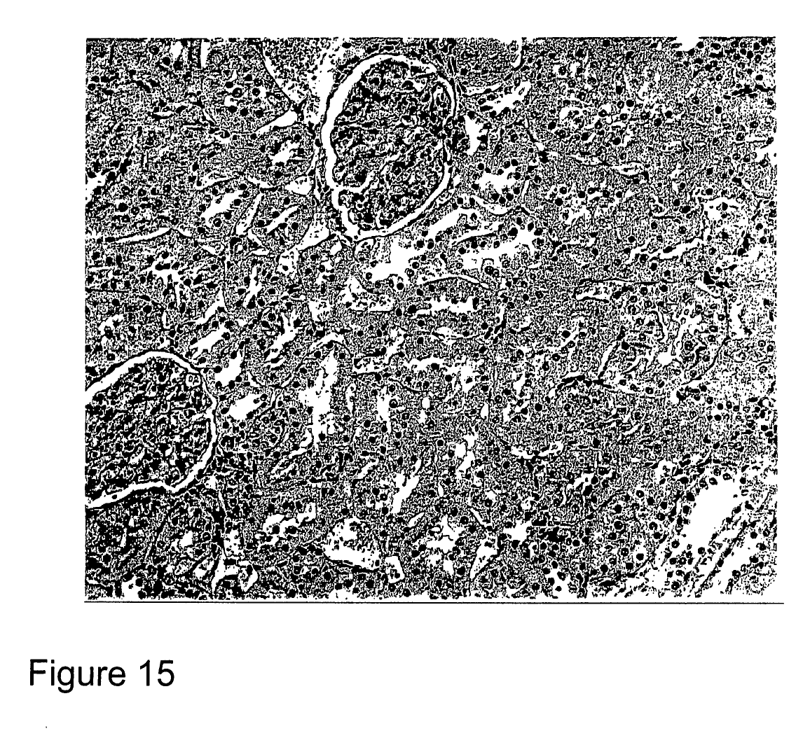

FIG. 15 shows the histological changes in a Sprague-Dawley rat with chronic renal failure after Aranesp (EPO) treatment (0.1 mg of Aranesp per kg) one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a normal glomerulus with delicate afferent vessel. No pathological signs were observed in the tubulointerstitium.

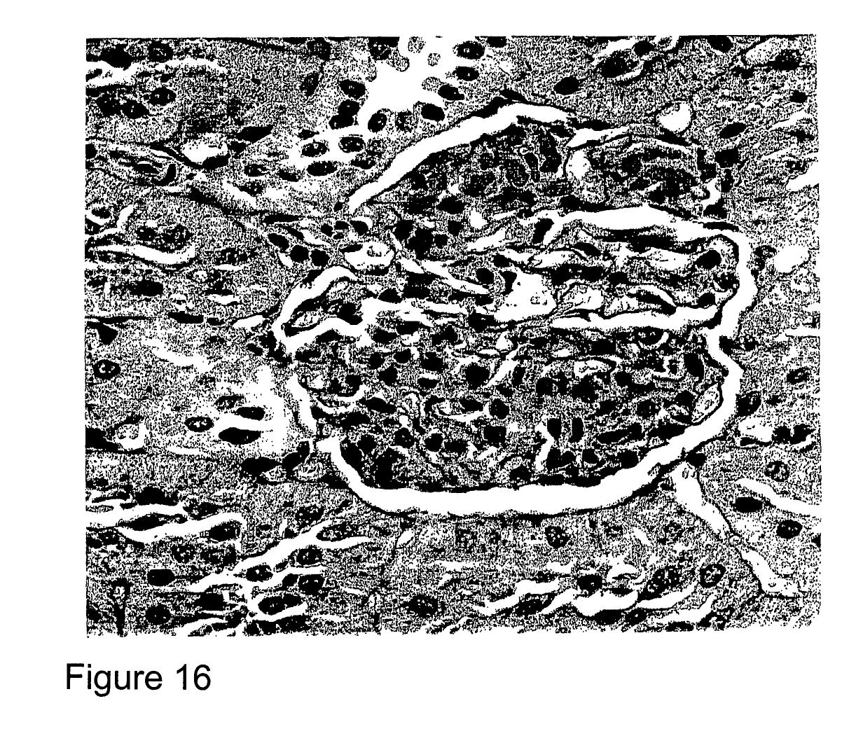

FIG. 16 shows the histological changes in a Sprague-Dawley rat with chronic renal failure after Aranesp (EPO) treatment (0.1 mg of Aranesp per kg) one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a normal glomerulus with delicate afferent vessel (630× magnification). No pathological signs were observed in the tubulointerstitium.

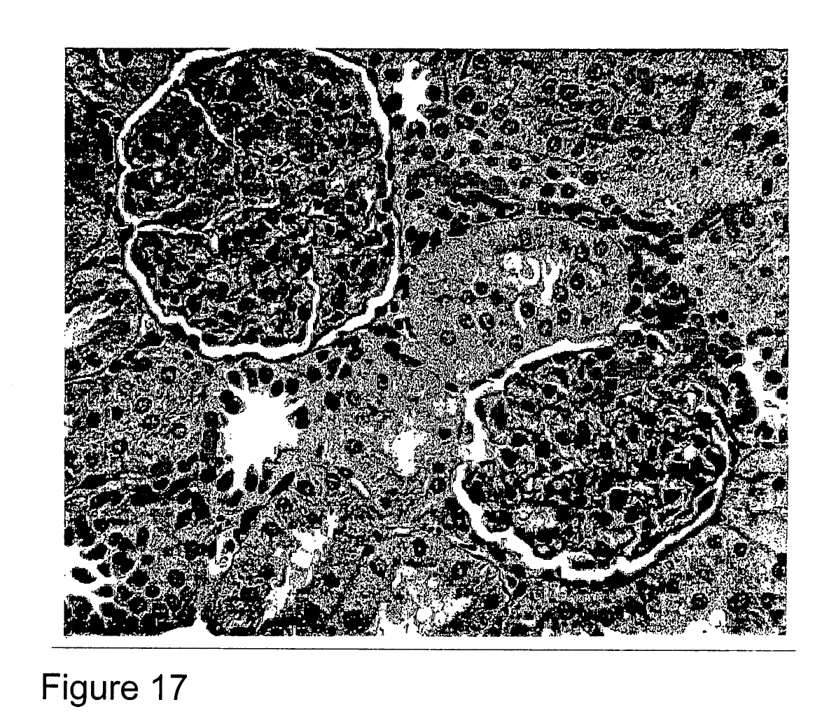

FIG. 17 shows the histological changes in a Sprague-Dawley rat with chronic renal failure after Aranesp (EPO) treatment (0.1 mg of Aranesp per kg) one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a normal glomerulus with delicate afferent vessel. No pathological signs were observed in the tubulointerstitium.

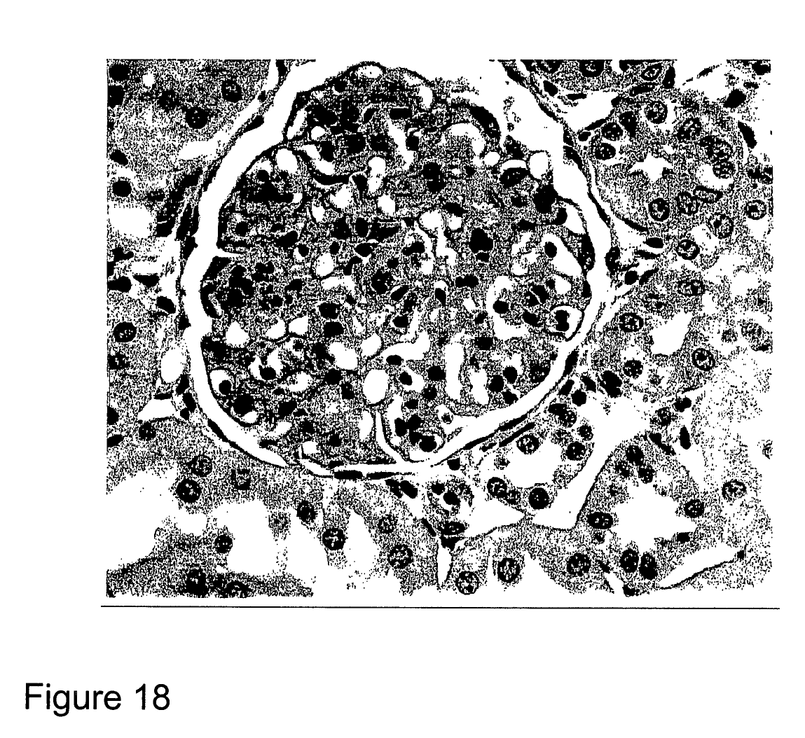

FIG. 18 shows the histological changes in a Sprague-Dawley rat with chronic renal failure after Aranesp (EPO) treatment (0.1 mg of Aranesp per kg) one time per week for a period of 6 weeks, beginning immediately after induction of chronic renal failure. The chronic renal failure was caused by removal of the right kidney and ligation of the segmental arteries supplying the upper and lower renal poles of the left kidney. The figure shows a normal glomerulus with delicate afferent vessel (630× magnification). No pathological signs were observed in the tubulointerstitium.

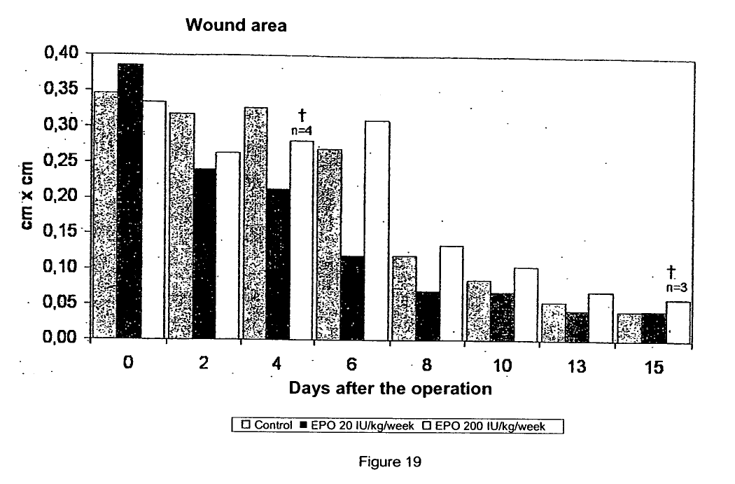

FIG. 19 shows the effect of EPO on the wound-healing process.

### EXAMPLE 1

Effect of EPO in Patients with Renal Anemia

The effect of erythropoietin in patients with renal anemia (Hb<10.5 g/dl) as a consequence of renal disease in the terminal stage (preterminal renal failure; creatinine clearance<35 ml/min) was investigated. 11 patients were treated intravenously or subcutaneously with erythropoietin in weekly doses averaging 5000 IU of rhEPO (recombinant human erythropoietin) for a period of at least 8 weeks. After erythropoietin treatment, the endothelial progenitor cells in the blood of the patients were investigated over a period of 20 weeks, the endothelial progenitor cells being analyzed with regard to number and differentiation status by flow cytometry and a culture test after 0, 2, 4, 6 and 8 weeks.

Circulating peripheral blood stem cells (CPBSC) represent a small population of cells that express both the CD34 antigen and the CD45 antigen. A test based on the ISHAGE guidelines has been developed to determine the number of CPBSC by flow cytometry (Sutherland et al., J. Hematother., 5 (1996), 213-226). Using this test, both the expression pattern of CD34 and CD45 cells and the morphology of the stem cells were determined. In this way, both the absolute number of CPBSC per μl and the content of CPBSC as a percentage of the total leukocyte count were determined.

FIG. 1 shows the results of an FACS analysis of circulating CD34+ stem cells on the basis of the ISHAGE guidelines.

FIG. 2 shows the number of CD34+ stem cells measured by FACS analysis over a period of 8 weeks.

Cell Culture Test

Peripheral blood mononuclear cells (PBMCs) were isolated by Ficoll density centrifugation from human blood samples in accordance with the method described in Asahara, Science, 275 (1997), 964-967. The cells were plated out on culture plates with fibronectin and maintained in EC basal medium. EC basal medium consists of EBM-2 basal medium (Clonetics Co.) and EGM-2 Quots (hEGF; GA-100 (gentamicin, amphotericin-B) FBS, VEGF, hFGF-B (w/heparin), R3-IGF-1, ascorbic acid, heparin). After 4 days of cultivation, nonadherent cells were removed by washing the plates. The remaining adherent cells were treated with trypsin and plated out once again. Thereafter they were cultivated for a further 3 days. Cells with the endothelial phenotype were identified by positive staining for two different endothelial markers on day 7 after isolation. These are Dil-labeled acetylated low density lipoprotein (acLDL-Dil) and Ulex europaeus aglutinin-1 (UEA-1). The results of this investigation are presented in FIG. 3.

The results show that erythropoietin is able to mobilize endothelial progenitor cells and to increase the number of circulating endothelial progenitor cells. In the process, functional deficits that occur in certain pathological states such as renal anemia are compensated. These results are presented in FIG. 4.

By means of flow cytometry it was found that the number of circulating CD34+ stem cells in patients with renal disease in the terminal stage corresponds to the number of circulating CD34+ stem cells in the blood of healthy subjects. After the erythropoietin treatment is started, the number of CD34+ stem cells in the bloodstream increases significantly by more than 50%. By using the cell culture assay, it was determined that, after treatment with erythropoietin, the number of cells that develop an endothelial phenotype increases dramatically. In one functional cell culture test, the greatly impaired ability of endothelial progenitor cells increased by a factor of greater than 3.

### EXAMPLE 2

Improved Wound Healing Through Systemic Use of rhEPO

FVB/N mice were anesthetized by inhalation anesthesia with isoflorane. The fur on the two rear limbs was removed using a depilatory lotion and disinfected with 70% alcohol. A sterile 4 mm disposable biopsy tissue punch was used to inflict a skin wound on the right flank of each of the mice. The opposite side served as internal control. Postoperative antibiotic cover with penicillin G (20,000 units/kg) was administered one time. Throughout the entire period of investigation, subcutaneous injections of the recombinant human erythropoietin analog Aranesp (0.1 μg/kg of body weight) were applied one time per week throughout the entire study period. The treatment began seven days before removal of the tissue punch. The results are presented in FIG. 8. They show that administration of EPO considerably accelerates the wound-healing process. FIG. 19 shows the effect of erythropoietin on wound healing. The figure shows that, when a standardized skin wound inflicted on mice using a tissue punch was treated with low-dosage erythropoietin (20 IU EPO/kg/week), it already closed completely after seven to eight days. In contrast, when the wound was treated with physiological salt solution (saline), it did not close completely until after thirteen to fourteen days. In the case of treatment of the experimental animals with high-dosage erythropoietin (200 IU EPO/kg/week), no acceleration of wound healing could be observed by comparison with the control group. Two of the experimental animals treated with high-dosage erythropoietin died during the observation period. The treatment with erythropoietin or physiological salt solution began on the day of the operation, after the skin wound was inflicted. Recombinant human erythropoietin was administered one time per week by s.c. (subcutaneous) injection (20 IU/kg EPO or 200 IU/kg EPO) (n=5 in each group).

### EXAMPLE 3

Reduction in the Progression of Chronic Renal Failure Through Erythropoietin Treatment

Eight-week-old Sprague-Dawley rats were anesthetized with ketamine (120 mg/kg) and Rompun (10 mg/kg). Their right kidney was removed on day 0 and was immediately fixed in formalin for histological examination. The segmental arteries supplying the upper and lower renal poles of the left kidney were ligated. Thereby renal infarction occurred in the corresponding kidney areas, and only the middle third of the kidney remained functional. One time per week, the rats received the erythropoietin analog Aranesp in a dose of 0.1 μg/kg of body weight or NaCl by s.c. injection for control purposes.

FIG. 10 shows the Kaplan-Mayer survival curves for both experimental groups. The animals treated with Aranesp have distinctly improved survival compared with the control animals treated with saline.

FIGS. 15 to 18 show that the renal tissue exhibits no pathological changes after treatment with erythropoietin, whereas severe pathological changes are visible after treatment with NaCl (compare with FIGS. 8 to 11). Further histological investigations revealed that a distinctly greater vessel density (CD31) can be observed in animals treated with Aranesp than in animals treated with saline (data not shown).

### EXAMPLE 4

Reduction in the Progression of Acute Renal Failure

Sprague-Dawley rats with a body weight of 250 to 300 g were used for this investigation. One of the experimental groups received Aranesp in a dose of 0.1 μg/kg of body weight one time on the day before induction of acute renal failure. The rats were anesthetized with ketamine (120 mg/kg of body weight) and Rompun (10 mg/kg). For comparison, there was used a group of experimental animals, each of which was given an s.c. injection of saline at the same time. By application of an arterial clamp to the right renal artery, the blood flow into the kidney was interrupted for 45 minutes. During this period, a left nephrectomy was performed. A sham operation was performed on a further control group. In this procedure, the abdomen was opened to expose the left renal artery, but the blood supply was not interrupted and the contralateral right kidney was removed. All animals were anesthetized for 60 min and killed 24 h after the operation.

In the animals treated with saline, the 45-minute ischemia with subsequent reperfusion of the remaining right kidney led to massive acute loss of renal function. This is reflected by the fact that the serum creatinine level increased by a factor of 7 (p<0.05). In contrast, the animals treated with the erythropoietin analog Aranesp exhibited only a four-fold increase in the serum creatinine levels one day after induction of damage by ischemia and reperfusion. No increase in retention levels was found in the animals subjected to left nephrectomy and a sham operation on the right kidney. The results are presented in FIG. 9.

### EXAMPLE 5

Reduced Differentiation Ability of Endothelial Progenitor Cells in Patients with Restricted Renal Function

The differentiation status of endothelial progenitor cells was analyzed by a culture test in 46 uremic patients as well as 46 healthy control subjects of matched age and sex. It was surprisingly found that the number of endothelial progenitor cells in this differentiation assay is significantly reduced in uremic patients compared with the healthy controls (FIG. 7). If mononuclear cells of a healthy subject are isolated and cultivated in the presence of serum of a uremic patient, the ability of these cells to differentiate to endothelial progenitor cells is reduced analogously (FIG. 6).

### EXAMPLE 6

Stimulation of the Differentiation Ability of Endothelial Progenitor Cells in Healthy Subjects

Four healthy young males were treated with 30 IU of epoetin beta per kilogram of body weight one time per week for a period of 8 weeks. The differentiation ability of their endothelial progenitor cells was determined in a culture assay, based on their adhesion ability and the two markers acLDL and UEA, before treatment of the subjects with rhEPO as well as weekly at 1, 2, 3, 4, 5, 6 and 7 weeks after treatment of the subjects with rhEPO. A relative increase of greater than 50% was observed in the EPCs.

1. The use of erythropoietin and/or derivatives for production of a pharmaceutical composition containing a dose of 1 to 90 IU/kg of body weight per week, preferably 1 to 45 IU/kg of body weight per week, for prevention or treatment of diseases, wherein the erythropoietin in this dose is suitable and designed for prevention or treatment of a human or animal patient exhibiting a) at least one dysfunction of endothelial progenitor cells, b) at least one cardiovascular risk factor such as hypertension, hypercholesterolemia, elevated asymmetric dimethylarginine (ADMA) levels, increased insulin resistance or hyperhomocysteinemia and c) at least one end-organ damage, namely left ventricular hypertrophy, microalbuminuria, cognitive dysfunction, increased thickness of the intima media in the carotid artery, proteinuria or a glomerular filtration rate of 30 to 80 ml/min.

2. The use of erythropoietin and/or derivatives for production of a pharmaceutical composition containing a dose of 1 to 90 IU/kg of body weight per week, preferably 1 to 45 IU/kg of body weight per week, wherein the erythropoietin in this dose is suitable and designed for cosmetic treatment of the human or animal body, especially for treatment of wrinkles, for strengthening of the connective tissue, for protection and tightening of the skin, for protection against harmful environmental effects, for treatment of age spots, for acceleration of reepithelialization, for acceleration of hair growth and/or as makeup foundation.

3. The use of erythropoietin and/or derivatives for production of a pharmaceutical composition containing a dose of 1 to 90 IU/kg of body weight per week, preferably 1 to 45 IU/kg of body weight per week, for production of a cosmetic preparation, especially for topical application, wherein the erythropoietin in this dose is suitable and designed for cosmetic treatment of the human or animal body, especially for treatment of wrinkles, for strengthening of the connective tissue, for protection and tightening of the skin, for protection against harmful environmental effects, for treatment of age spots, for acceleration of reepithelialization, for acceleration of hair growth and/or as makeup foundation.

4. The use of erythropoietin and/or derivatives for production of a pharmaceutical composition containing a dose of 1 to 90 IU/kg of body weight per week, preferably 1 to 45 IU/kg of body weight per week, and/or a mixture of endothelial progenitor cells with at least one cell population usable for cell therapy, wherein the erythropoietin in this dose is suitable and designed for regeneration of tissues or vessels in a human or animal body, and wherein the mixture has been brought into contact with erythropoietin in vitro prior to application.

5. The use of erythropoietin and/or derivatives for production of a pharmaceutical composition containing a dose of 1 to 90 IU/kg of body weight per week, preferably 1 to 45 IU/kg of body weight per week, and/or a mixture of endothelial progenitor cells with at least one cell population usable for cell therapy, wherein the erythropoietin in this dose is suitable and designed for regeneration of tissues or vessels in a human or animal body, and wherein the erythropoietin is administered before, after or simultaneously with application of the mixture.

6. The use of erythropoietin and/or derivatives for production of a pharmaceutical composition or of a kit containing a dose of 1 to 90 IU/kg of body weight per week, preferably 1 to 45 IU/kg of body weight per week, and/or at least one chemical, thermal, mechanical or biological agent, especially a pharmacological active ingredient, for production of a pharmaceutical composition or of a kit containing erythropoietin in this dosage and the at least one chemical, thermal, mechanical or biological agent, for prevention or treatment of diseases, wherein the erythropoietin in this dose is suitable and designed for sequential, timed successive or simultaneous application of the erythropoietin with the at least one chemical, thermal, mechanical or biological agent.

7-8. (canceled)

9. The use of erythropoietin and/or derivatives for production of a pharmaceutical composition containing a dose of 1 to 90 IU/kg of body weight per week, preferably 1 to 45 IU/kg of body weight per week, wherein the erythropoietin in this dose is suitable and designed for prevention or treatment of diseases, wherein the disease is hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, renal function restrictions with glomerular filtration rates of 30 to 80 ml/min, microalbuminuria, proteinuria, or wounds and sequelae thereof.

10. The use of erythropoietin and/or derivatives for production of a pharmaceutical composition containing a dose of 1 to 90 IU/kg of body weight per week, preferably 1 to 45 IU/kg of body weight per week, for production of a kit containing erythropoietin, endothelial progenitor cells and at least one cell population usable for cell therapy, wherein the erythropoietin is preferably present in low dosage.

11-14. (canceled)

15. The use of erythropoietin in a low dosage of 1 to 90 IU/kg of body weight per week for the therapy of pathological states or diseases of the human or animal body associated with a dysfunction of endothelial progenitor cells, and wherein the pathological states or diseases associated with a dysfunction of endothelial progenitor cells are hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, renal function restrictions with glomerular filtration rates of 30 to 80 ml/min, microalbuminuria, proteinuria, elevated ADMA levels or wounds and sequelae thereof.

16-18. (canceled)

19. The use of erythropoietin in a low dose, especially of 1 to 90 IU/kg of body weight per week, for the therapy of hepatic disorders such as hepatitis, cirrhosis of the liver, acute or chronic liver failure, bone and cartilage disorders or lesions, mucous membrane disorders or lesions, especially in the gastrointestinal tract, Crohn's disease, ulcerative colitis, renal function restrictions with glomerular filtration rates of 30 to 80 ml/min, microalbuminuria, proteinuria, elevated ADMA levels or wounds and/or sequelae thereof.

20-31. (canceled)

32. The use of erythropoietin for production of a transplantable endothelial preparation.

33-38. (canceled)

39. A pharmaceutical composition for stimulation of endothelial progenitor cells, for stimulation of the formation of endothelial tissue, for stimulation of vasculogenesis and/or for treatment of diseases or pathological states associated with a dysfunction of endothelial progenitor cells, comprising erythropoietin and/or a derivative, an analog, a modification or a mutein thereof as the active ingredient as well as at least one further active ingredient selected from the group comprising VEGF, PIGF, GM-CSF, an ACE inhibitor such as enalapril, ramipril or trandolapril, an AT-1 blocker such as irbesartan, lorsartan or olmesaratan, an HMG-CoA reductase inhibitor and an NO donor, preferably in a low dose, especially of 1 to 90 IU/kg of body weight per week.

40. A pharmaceutical composition for prevention and/or therapy of hepatic disorders such as hepatitis, cirrhosis of the liver, acute or chronic liver failure, bone and cartilage disorders or lesions, ligament and tendon disorders or lesions, mucous membrane disorders or lesions, especially in the gastrointestinal tract, Crohn's disease, ulcerative colitis, renal function restrictions with glomerular filtration rates of 30 to 80 ml/min, microalbuminuria, proteinuria, elevated ADMA levels or wounds and sequelae thereof, comprising erythropoietin and/or a derivative, an analog, a modification or a mutein thereof as the active ingredient, preferably in a small dose, especially of 1 to 90 IU/kg of body weight per week.

41-44. (canceled)

45. A kit containing erythropoietin, endothelial progenitor cells and at least one cell population usable for cell therapy, wherein the erythropoietin is preferably present in low dosage.

46. The use of erythropoietin and/or derivatives for production of a pharmaceutical composition or of a kit containing a dose of 1 to 90 IU/kg of body weight per week, preferably 1 to 45 IU/kg of body weight per week, for prevention or treatment of diseases of the human or animal body, wherein the erythropoietin in the said low dose is suitable and designed for improving, especially for promoting and/or accelerating, the integration of a mechanical or biological agent, especially an endoprosthesis, especially an implant, for example a tooth implant, a tooth replacement, a bone implant, a bone replacement, especially a joint prosthesis, a ligament/tendon replacement, such as the cruciate ligaments, or a solid organ into the implant or the body structures surrounding the endoprosthesis.

47-48. (canceled)

49. A kit containing erythropoietin in a dose of 1 to 90 IU/kg of body weight per week, preferably 1 to 45 IU/kg of body weight per week, an endoprosthesis and if necessary a cell therapeutic, preferably endothelial progenitor cells or other cell populations usable for cell therapy.

50. The use of erythropoietin and/or derivatives for production of a pharmaceutical composition containing a dose of 1 to 90 IU/kg of body weight per week, wherein the erythropoietin in this dose is suitable and designed for prevention or treatment of insulin resistance.

51. (canceled)

52. The use of erythropoietin in a small dose of 1 to 90 IU/kg of body weight per week for therapy of insulin resistance.

53. A pharmaceutical composition for prevention and/or therapy of insulin resistance, comprising erythropoietin and/or a derivative, an analog, a modification or a mutein thereof as the active ingredient, preferably in a small dose, especially of 1 to 90 IU/kg of body weight per week.
